(19) United States
(12) Patent Application Publication
(10) Pub. No.: US 2012/0296090 A1
(43) Pub. Date: Nov. 22, 2012

(54) Drug Repositioning Methods For Targeting Breast Tumor Initiating Cells

(75) Inventors: Stephen TC Wong, Houston, TX (US); Hong Zhao, Houston, TX (US); Guangxu Jin, Houston, TX (US)

(73) Assignee: The Methodist Hospital Research Institute, Houston, TX (US)

(21) Appl. No.: 13/439,626

(22) Filed: Apr. 4, 2012

Related U.S. Application Data
(60) Provisional application No. 61/561,666, filed on Nov. 18, 2011.
(60) Provisional application No. 61/471,559, filed on Apr. 4, 2011.

(51) Int. Cl. G06F 19/00; C07D 305/14; C07C 233/15; C07D 413/12

(52) U.S. Cl. 544/119; 549/510; 564/218; 702/19

(57) ABSTRACT

Disclosed are systems biology-based methods for repositioning known pharmaceutical compounds to new indications, through the identification of network-based signatures. In particular, the invention provides new and useful methods for selecting drugs or combinations of drugs (and preferably previously-approved drugs) for use in new therapeutic indications. Also disclosed are methods for identifying anti-breast tumor initiating cell (TIC)-based therapeutics from within populations of target compounds. In illustrative embodiments, the invention provides methods and computer programs for the repositioning of FDA-approved pharmaceutical compounds to new indications using network-based signature analysis coupled with conventional in vitro and in vivo testing of identified drug candidates. The invention also allows identification of drugs or drug combinations for treating unmet medical needs including, for example, “orphan” diseases.

### CROSS-REFERENCE TO RELATED APPLICATIONS

The present application claims priority to U.S. Provisional Patent Application Nos. 61/471,559 and 61/561,666, filed Apr. 4, 2011 and Nov. 18, 2011, respectively (each co-pending); the entire contents of each of which is specifically incorporated herein in its entirety by express reference thereto.

### STATEMENT REGARDING FEDERALLY SPONSORED RESEARCH OR DEVELOPMENT

This invention was made with government support under Grant No. U54CA149196 from the National Institutes of Health. The government has certain rights in the invention.

### NAMES OF THE PARTIES TO A JOINT RESEARCH AGREEMENT

Not Applicable.

### BACKGROUND OF THE INVENTION

1. Field of the Invention

The present invention relates to the identification and development of drug regimens and treatment modalities for breast cancer in a patient. In particular, a systems biology method has been developed to reposition known pharmaceutical compounds by identifying network-based signatures, and to identify anti-breast Tumor Initiating Cells-(breast TICs)-specific therapeutic compounds. In certain illustrative embodiments, methods are provided for repositioning known pharmaceutical compounds (and particularly those already approved by the Food and Drug Administration [FDA]) to network-based signatures of other cancers (including, for example, breast TICs), and for testing the compounds identified thereby through in vitro and/or in vivo assays (including, for example, those assays specific for anti-cancer, and particularly, anti-TIC activity).

2. Description of Related Art

Breast Cancer

Excluding cancer of the skin, breast cancer is the most common cancer diagnosed in American women (1 in 8 women; about 13%) and is the second leading cause of cancer deaths among women. Recent clinical data, as well as experimental evidence in both mouse mammary tumors and human xenograft models, support the existence of a subpopulation of cancer cells present in the original tumor that are greatly enriched in residual cancers after conventional systemic therapies. These residual cancer cells are characterized by their intrinsic resistance to chemotherapy and relative growth quiescence. However, a discreet subset of these residual cells possesses enhanced self-renewal capacity, as well as the ability to form tumors upon transplantation. These residual tumor-initiating cells (TICs) (a.k.a. “cancer-stem cells” [CSCs]) may therefore be responsible for tumor growth, maintenance, resistance to treatment, and disease relapse. These findings fundamentally modify traditional conceptual approaches to oncogenesis, and have dramatic implications for breast cancer prevention, treatment, and drug development.

Signaling Pathways

Signaling pathways have been a key target in cancer therapy. One of the challenges is that most known signaling pathways often capture only a small fraction of critical genes or proteins relevant to a particular type of cancer. The conventional approaches that focus on drug-targets in the incomplete pathways may ignore many essential pathways that are responsible for the downstream effect on gene transcription and inevitably fail to understand the real effect of drugs.

Drug Repositioning

Little research has been done to address the huge opportunities that may exist to reposition existing approved or generic drugs for alternate uses in cancer therapy. Additionally, there has been little work on strategies to reposition experimental cancer agents for testing in alternate settings that could shorten their clinical development time. Progress in each area has lagged in part due to the lack of systematic methods to define drug off-target effects (OTEs) that might affect important cancer cell signaling pathways.

During the past several years, there has been a surge of interest in drug repositioning. Both pharmaceutical and biotech companies have recognized the advantages of repositioning, and activity in the area has increased dramatically. There are a number of examples in which serendipity or directed efforts have led to successful launches in new indications. Ideas for repositioning can come from serendipitous observations (for example, sildenafil); from novel, informed insights (for example, duloxetine); or from technology platforms established to identify repositioning opportunities (for example, Zalicus/CombinatoRx Inc.'s cHTS™ drug discovery technology system; Cambridge, Mass., USA). Once the repositioning idea has been generated, and the proposed approach scientifically validated, then a commercially-viable target product profile for a candidate can be generated, and a search will be conducted to identify compounds having the desired characteristics. So far, the unique challenge associated with conventional repositioning strategies is the increased demand for creative approaches to systemically generate potential repositionable drug candidates.

The study of drug repositioning has so far been limited to the “on-target repositioning” that applies a drug's known pharmacological mechanism to a different therapeutic indication; for example, comparing the structural similarities of small molecules (Keiser et al., 2009; Miller, 2002) or known side effects (Campillos et al., 2008). In contrast, “off-target repositioning” attempts to describe the pharmacological mechanisms still unclear for known molecules. A number of approaches have recently been developed for off-target repositioning by using gene signatures (Lamb et al., 2006; Lamb, 2007; Sirota et al., 2011; Sardana et al., 2011), i.e., subsets of genes, or drug-similarity network (Iorio, 2010) identified in the cancer transcriptional profiles following drug treatment. One common limitation of these methods is that they do not include the disease-specific prior knowledge or known mechanisms in the off-target repositioning process, so that they can be used to find similarities between the drugs but not the preference between them. Thus, there is a need to develop new methods that incorporate prior knowledge of specific diseases to improve the precision of off-target drug repositioning.

A primary challenge of off-target repositioning is to address the OTEs of a drug on the proteins downstream in the signaling pathways and the genes that are regulated by those proteins. As an example, in breast cancer raloxifene, tamoxifen, and fulvestrant are the pioneering drugs targeting the estrogen receptor (ER) (Wishart, 2008). The targeted proteins, however, often generate downstream effects on the linked signaling proteins and ultimately exert unexpected off-target effects on cancer transcription (Keiser and Hert, 2009; Feyen et al., 2008; Creighton et al., 2008). Creighton et al showed that tamoxifen together with estrogen deprivation (ED) can shut down classic estrogen signaling and activate alternative pathways such as HER2, which can also regulate gene expressions. The unexpected downstream signaling proteins and altered cancer transcription can be considered as the off-targets of the treated drugs.

Work has been conducted to address the off-targets using biomarkers or gene signatures (Lamb et al., 2006; Lamb, 2007; Sirota et al., 2011; Sardana et al., 2011; Iorio, 2010; Wishart, 2008; Keiser and Hert, 2009; Feyen et al., 2008; Creighton et al., 2008). Although the methods on gene signatures are able to identify which genes are changed during the treatment of a drug, they cannot explain the associations between the expression changes of the genes and the OTEs on these genes of the drug in terms of the pathway mechanism of the disease. Moreover, these methods also fail to identify frequently changed genes, which were not considered in the gene signatures.

In summary, existing strategies used in drug repositioning have numerous drawbacks, which have limited their effectiveness in generating drugs or drug combinations suitable for new medical indications. These drawbacks are mostly related to the fact that drug repositioning has been drug oriented (to find new therapeutic area for the old drug) rather than disease oriented (to find new therapies based on old, approved drugs). What is needed in the art are new and useful methods for identifying drug candidates for the treatment of diseases in human and animal populations.

### BRIEF SUMMARY OF THE INVENTION

The present invention overcomes these and other inherent limitations in the art by providing, in a general sense, a systems biology-based methodology useful for the identification and repositioning of drugs (particularly known drugs, and more preferably still, already FDA-approved drugs) through the creation and application of network-based signatures. In illustrative embodiments, such methods have been used to identify repositionable drugs that are also suitable for anti-breast tumor initiating cells (breast TICs) therapies.

The present invention relates to methods for drug repositioning. More particularly, this invention relates to methods for selecting approved drugs or combinations of approved drugs for use in new therapeutic indications. This approach is situated in a cross section between drug repositioning and disease treatment by combinations of drugs with additive or synergistic action. In certain embodiments, the invention facilitates the repositioning of known drugs to new indications, or defines drugs or drug combinations for treating unmet medical needs, such as “orphan” diseases and the like

While many strategies have been implemented by pharmaceutical companies to identify new drugs (such as screening combinatorial libraries for chemical pharmacophores, and high throughput screening of combinatorial libraries of molecules to determine their activity for a selected validated target or disease relevant phenotypical endpoint.), these approaches have produced limited results in identifying new uses for existing drugs, and the identification of new pharmacological uses of existing compounds, which have not yet been approved for human or animal use.

The exemplary drug repositioning methods described herein permit for the first time, a facile integration of a variety of diverse biological information (including, for example, gene expression profiles, protein interaction networks, and signaling pathways, etc.) into a single, unified process to identify network-based signatures useful for identifying drugs suitable for repositioning to other medical indications and/or therapies. The methods disclosed herein have been applied in particular to the identification of new drugs for treating breast TICs. Using the computational methods described herein, thousands of known compounds (including many already FDA-approved drugs) have now been mapped to a network-based signature generated for breast TICs. From these analyses, a total of twenty-one existing (and FDA-approved) drugs have now been identified as repositionable candidates for anti-breast TIC-based therapies. To demonstrate the facility of the method in generating meaningful subsets of highly-relevant candidates, the 21 identified drugs were tested (both in vitro and in vivo) for their ability to inhibit breast TICs. In a particular illustrative embodiment, these methods identified three repositionable drugs: sunitinib, dasatinib and chloroquine that were subsequently shown in vivo and in vitro to be effective in inhibiting breast TICs (see e.g., FIG. 3). One of the identified repositioned drugs, dasatinib, has now been employed in a human Phase II clinical trial for breast TICs-specific anti-cancer therapy. The drug repositioning methods described herein can be cost-effectively applied to identifying repositioning candidate drugs, and permit the rapid translation of repositioned drugs into subsequent clinical trials.

Using the disclosed methods, the inventors have identified a tumorigenic gene signature based upon profile features in common in CD44⁺/CD24^{-/low} cells vs. all other tumor cell subpopulations, and in cancer-derived mammospheres vs. bulk tumor “tumor-initiating cells” (TICs) (CD44^{hi}/CD24^{-/low}) (FIG. 4A and FIG. 4B). Through the use of bioinformatic systems model-based approaches, which exploit the known protein-protein interactions, signaling pathways and extant drug information on more than 6000 available drugs, the inventors successfully mapped known drugs to critical pathways identified in the tumorigenic signature to target breast TICs (FIG. 5).

The bioinformatics systems-based methods described herein also identified a further subset of ten drugs that were subsequently tested in vitro and in vivo for their ability to inhibit breast TICs. These in silico results were first confirmed by in vitro studies that evaluated the effects of these repositioned drugs on mammosphere-forming efficiency. In two “stem cell like” cell lines, SUM159 and BT549 treated with 10 nM and 1 μM of chloroquine, a dramatic reduction in MSFE was noted after a single treatment dose. Next, limiting dilution transplantation assays were performed, whereupon SUM159 cells were treated with 1 chloroquine overnight and then transplanted the next day 200,000 cells/mouse (single sided). A reduction in tumor initiation rate was observed in only 1 of 12 xenografts growing within 10 weeks with chloroquine treatment vs. 9 of 12 in the vehicle-treated control group.

The inventors have developed an approach for systemically generating repositioned drug candidates for targeting breast TICs that promises to fundamentally change the way in which drugs are identified and selected for treatment of breast cancer. By exploiting a combination of protein-protein interaction networks, signaling pathways, and a DrugMap Central database, the inventors were able to decipher the TICs signaling network, expand the network with repositioned drug targets, and map about 2,000 additional known (and FDA-approved) drugs to the signaling network. With the gene expression profiles for breast cancer TICs from patient samples, the TICs repositioning ideas were generated through the integration of mining these existing data with a new technology tool (i.e., cancer signaling bridges; CSBs), to expend the existing signaling pathways (CSB, FIG. 1), unique human-tumor-in-mouse xenografts and high content mammosphere formation efficiency assay to validate the drugs on inhibiting TICs in vitro and in vivo.

Compared to the traditional singular ways that have been reported for successful drug repositioning (i.e., serendipitous observations; “informed insights,” etc.); and the existing technology-based platforms that have been established to identify repositioning opportunities, the present invention offers significant advantages by combining the “informed insights” approach with a new technology platform-based computational approach to produce a robust and reproducible method for identifying target candidates for drug repositioning.

In sharp contrast to conventional methods that study drug targets directly, the present methods exploit CSBs to investigate the often-ignored downstream drug effects transmitted from expanded signaling pathways to cancer related genes or proteins. The inventors have successfully demonstrated that CSBs may be employed to comprehensively understand the total drug effect, and to reposition extant anti-cancer drugs for specific new cancer indications.

In a further improvement over existing methods, the present invention also adapts “wet lab” pre-clinical validation procedures, which permit the rapid translation of repositioned drugs into suitable clinical trials. In situations where all of the screened drugs are already FDA-approved, this method facilitates expedited enrollment of drugs into Phase II clinical trials, and thereby reduces the time, effort, and expense associated with de novo drug development. This inventive feature therefore increases the potential for improved patient quality-of-life, enhances clinical outcomes, and reduces the overall healthcare expense for cancer patients.

A further object of the invention is a method of producing a pharmaceutical compound from a population of candidate drugs that were identified using one or more of the methods disclosed herein. Similarly, a further object of the invention is a method of treating a disease, dysfunction, disorder, or abnormal condition in an animal (and preferably in a human), or ameliorating one or more symptoms of such, that generally involves providing to the animal an effective amount of at least a first drug identified from among a population of candidate drugs.

The present invention also provides compounds that are suitable for the treatment of a variety of diseases, including, without limitation, neurologic disorders, psychiatric disorders, cancers, autoimmune diseases, cardiovascular diseases, hematological diseases, metabolic diseases, and the like. In particular exemplary embodiments, compounds are identified for the treatment of breast cancer, and in particular, triple negative breast cancers, and other hyperproliferative disorders involving the action of breast tumor initiating cells, including for example, brain metastatic breast cancers.

In certain embodiments, the invention provides methods of identifying appropriate drugs or drug combinations for treating rare, orphan diseases (i.e., rare disease which has a very low prevalence in the population), or common diseases with unmet therapeutic need (i.e., endemic and pandemic diseases in developing nations such as tuberculosis, typhoid, malaria, etc.). Such conditions may include, without limitation, one or more neuropathies, diabetic and drug-induced neuropathies, amyotrophic lateral sclerosis (ALS), stroke, Parkinson's disease, Huntington's disease, Alzheimer diseases, dementia, schizophrenia, bipolar disorder, major depression, diabetes, cardiac diseases, rare genetic disorders, and the like.

In one embodiment, the invention provides a method for repurposing a known pharmaceutical compound to a new treatment protocol. In an overall and general sense, such method generally includes one or more of the following steps: a) identifying a pharmaceutical compound, wherein the pharmaceutical compound is an untested compound, a compound that has failed in clinical development, or a drug that has been approved for use in another distinct treatment protocol; b) creating a computer model for one or more cancer signaling bridges; c) performing computer simulations using the computer model with data obtained from one or more in vitro or in vivo assays, one or more animal models of disease, or one or more pre-clinical or clinical trials of the pharmaceutical compound in at least a first human subject; and d) identifying a new treatment protocol for the known pharmaceutical compound based upon the results obtained from c).

In particular embodiments, the new treatment protocol may include at least one component such as a new indication for the known drug, a new drug for a known indication, a new dosing regimen for a known drug in new or existing dosing regimens, a new combination of drugs for treating an existing or new indication, a new patient population for use of the drug or combination of drugs, and/or the use of one or more biomarkers represented in the computer model to differentiate between responders and non-responders in a patient population.

In illustrative examples, the new treatment protocol is a new indication for the treatment of a human disease, dysfunction, or disorder, with the treatment of cancers, neurological, hematological, and metabolic disorders being particularly preferred.

In one illustrative example, the invention provides new methods for treating breast cancers, including triple negative breast cancer, and other hyperproliferative disorders caused by breast tumor initiating cells, including metastatic breast cancer . . . is breast cancer.

In the practice of the invention, treatment of the new indication with the repurposed drug may result in reduced drug toxicity, increased drug efficacy, or a combination of both, in a recipient patient when compared to the use of an existing known drug already approved for treatment of such a indication.

Preferably, the compounds of the invention include known pharmaceutical compound, which are already approved by the FDA for administration to a human, or are currently in one or more phases of clinical trials to certify the drugs for human or animal administration.

The invention also provides a method for identifying a candidate drug for the treatment of a selected disease or condition in an animal in need thereof. Such method generally includes the steps of 1) selecting a disease or condition to be treated; 2) creating a dynamic model of the disease; 3) performing in silico screening of a population of drugs, one or more of which is approved for other diseases or conditions that target one or more biological pathways implicated in the model to identify one or more candidate drugs; 4) testing the one or more candidate drugs selected in 3), either alone or in one or more combination(s) of drugs, in a biological model of the disease; and 5) identifying from within the candidate drugs tested in 4), one or more bioeffective drugs, or combinations thereof, suitable for the treatment of the selected disease or condition. Such methods may be useful in the treatment or amelioration of one or more symptoms of diseases or conditions such as neurologic diseases and disorders; psychiatric diseases and disorders; autoimmune diseases and disorders; cardiovascular diseases and disorders; metabolic conditions, metabolic dysfunctions, metabolic diseases; neuropathic dysfunctions, neurological disorders, hematological diseases and disorders; neuromuscular disease or dysfunction; or one or more hyperproliferative disorders such as cancer, or any combination thereof. In such a method, the dynamic model of the disease or condition may be created, for example, by compiling experimental data that describes or characterizes the phenotype of the disease, disorder, or condition at a genomic, biochemical, cellular or organismal level.

In another embodiment, the invention provides a computer program product that includes a computer-readable medium containing instructions that permit a computer to implement one or more of the processes described herein, such as, for example, a process that includes: a) identifying a pharmaceutical compound, the pharmaceutical compound corresponding to a drug that has failed in clinical development or an approved drug; b) creating a computer model for pharmacokinetics and pharmacodynamics of the drug is created based on data of effects of the drug administered in vitro or in vivo to determine the physiological effect of the drug on a disease; c) adjusting the computer model based upon results of computer simulations from at least one pre-clinical or at least one clinical trial; d) determining a new treatment protocol to salvage the failed drug or the approved drug based on the results of computer simulation results; and e) displaying the new treatment protocol in an output window.

The invention also provides a system for off-target repositioning of a pharmaceutical compound. Such a system generally includes identifying a pharmaceutical compound, wherein the pharmaceutical compound is a drug that has failed in clinical development or an approved drug; a computer model for pharmacokinetics and pharmacodynamics of the drug is created based on data of effects of the drug administered in vitro or in vivo to determine the physiological effect of the drug on a disease; a model adjustor that adjusts the computer model based upon results of computer simulations from at least one pre-clinical or at least one clinical trial; a treatment protocol generator that results in the off-target repositioning of the pharmaceutical to a new indication or the identification of a new treatment protocol for the repositioned compound.

In illustrative systems, the new treatment protocol may be based upon at least one component selected from a new regimen, a new drug combination, a new disease or condition, a new patient population, or a use one or more biomarkers represented in the computer model to differentiate between responders and non-responders in the patient population.

In particular embodiments, the computer model involves analysis of one or more cancer signaling bridges in an animal, and in a human in particular, wherein the analysis of such cancer signaling bridges is useful in identifying one or more candidate drugs for the treatment or amelioration of symptoms of at least one type of cancer (and preferably breast cancer) in a patient in need of such treatment.

In the practice of the invention, the identified candidate drugs may be new, untested drugs, drugs tested for one or more failed treatments, or drugs already approved and used for one or more indications in human. The repurposing of such compounds may involve “on target” repositioning or, in preferred embodiments, “off-target” repositions. In particular, the identified candidate drug may be one that is already employed in the treatment of one cancer indication, but heretofore unknown and/or untested or unused for the treatment of a second, distinct cancer indication. For example, a drug candidate identified by one or more of the disclosed methods may already be an FDA approved drug for the treatment of prostate cancer, but may not yet have been tested or approved for the treatment of a second, unrelated type of cancer. In some applications, cancer drugs approved for one type of cancer will be repositioned to treatment of breast cancer, and in particular, triple-negative breast cancer.

In illustrative embodiments, the inventors have identified a subset of known FDA-approved drugs using the disclosed methods that are likely useful in the treatment of other cancer types. Exemplary members of that subset include, but are not limited to, L-glutamic acid, L-proline, sorafenib, rifabutin, adenosine, simvastatin, naltrexone, clodronate, auranofin, thalidomide, atorvastatin, arsenic trioxide, saquinavir, glucosamine, procaterol, clenbuterol, pranlukast, aminone, sunitinib, dasatinib, chloroquine, and any combinations thereof.

In certain embodiments, the data obtained from one or more in vitro or in vivo assays includes data obtained from one or more gene expression profiles, one or more protein expression profiles, one or more protein:protein interaction networks, one or more signaling pathways, one or more cancer signaling bridges, or any combination thereof. Such data may be indicative of drug targeting to one or more of proteins selected from the group consisting of TNF, KDR, IKBKB, Notch, hedgehog, wnt, P13K, FYN, SLC25A6, HSP90AA1, and EPRS, or to one or more genes that encode the one or more proteins, or any combination thereof.

In some aspects of the invention, the known pharmaceutical compounds identified as suitable repositionable candidates may have relatively low or relatively insignificant “off target” effects on one or more of the gene(s) or protein(s) downstream in at least one of the metabolic or signaling pathway(s) for the new indication for which a candidate drug is being sought.

In the practice of the invention, one or more computer models may be developed that employ one or more bioinformatic systems model-based approaches to exploit one or more of the following: a) known protein-protein interactions, b) one or more steps in a signaling pathway that controls one or more steps in the metabolic pathway giving rise to the new indication being treated, or c) extant pharmacological information, to increase the likelihood of identifying a compound that is effective in treating the new indication for which a candidate drug is being sought. In some embodiments, the computer model of the new indication may be useful in designing one or more pre-clinical or clinical trials to testing the effectiveness of the repositioned pharmaceutical compound in vivo. While the analyzed data may be stored on a local computer or data server, in commercially-relevant embodiments, the inventors contemplate centralized storage of the data in a database that is accessible to one or more operators employing the computer modeling methods as disclosed herein. Such databases may be accessible through convenient modes in the art, such as via the Internet, via a subscription-based model, or by the use of one or more web-based/server applications.

In certain embodiments, the computer model for the new indication or new protocol may be obtained by combining information and data available from a number of sources into a logically-supported pathway that is involved in disease genesis and/or progression.

The methods of the invention may also further optionally include one or more additional steps, such as identifying one or more pathways of the model of the disease or condition that could be modulated or blocked by the action of one or more identified candidate drugs, or alternatively, the identification and/or selection of one or more approved drugs that are known to interact with at least one target or with at least one step in a metabolic or signaling pathway that is included in, or that defines a key step in, the dynamic model of the disease or condition for which a repurposed drug is being sought.

In a further aspect, a method of producing a pharmaceutical compound, or a combination of two or more pharmaceutical compounds is provided, particularly useful in the treatment, or amelioration of one or more symptoms of, a particular disease, dysfunction, or abnormal condition in an animal (and preferably in a human). In an overall and general sense, such a method may include one or more of the following steps: a) identifying a candidate drug or a combination of candidate drugs effective for treating the selected disease, dysfunction, or condition; and b) producing the candidate drug or the combination of candidate drugs in a pharmaceutically acceptable form. In exemplary embodiments, the identified candidate drugs may be effective in the treatment of breast cancer, or in the inhibition of breast tumor initiating cells in vivo or in vitro.

### BRIEF DESCRIPTION OF THE DRAWINGS

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1 shows an illustration of cancer-signaling bridges (CSBs). The CSBs are specific instances of network motifs in a protein-protein interaction network. Each CSB contains three or four proteins that are connected together by protein-protein interactions. CSB is able to connect a signaling pathway with cancer-related genes or proteins by its component proteins;

FIG. 2A, FIG. 2B, and FIG. 2C illustrate an exemplary flowchart for drug repositioning in accordance with the present invention;

FIG. 3 describes the effects of vehicle, sunitinib, dasatinib, and chloroquine alone on human breast cancer xenograft mammosphere formation efficiency;

FIG. 4A and FIG. 4B show network-based signatures for drug repositioning obtained through application of a CSB model in accordance with one aspect of the present invention to the gene expression profiles of breast TICs derived from CD44+/CD24−/low breast cancer cells and mammospheres (MS) cells;

FIG. 5 shows a schematic diagram of TICs and the site of action of repositioned drugs identified in the practice of the present invention;

FIG. 6A, FIG. 6B, FIG. 6C, and FIG. 6D show aspects of the CSB-BFRM model in accordance with one aspect of the present invention;

FIG. 7A, FIG. 7B, FIG. 7C, FIG. 7D, and FIG. 7E show CSBs and their role in the study of cancer and in the discovery of new cancer drugs. FIG. 7A shows CSBs extend the signaling proteins to cancer proteins; FIG. 7B shows linked cancer types of CSBs; FIG. 7C illustrates known anti-cancer drugs targeted on the proteins for signaling pathways, CSBs, and cancer; FIG. 7D illustrates that extended proteins by CSBs are more likely to be targeted by anti-cancer drugs than non-extended ones (signaling proteins: P<10⁻⁵, cancer proteins: P<10⁻¹⁴, Fisher's exact two-tailed test); FIG. 7E illustrates the overall effects on protein sets evaluated by E-scores. For known anti-cancer drugs, they had significantly higher effects on cancer protein set than those of signaling pathways and CSB proteins (P<10⁻²⁰, Mann-Whitney U test);

FIG. 8A, FIG. 8B, FIG. 8C, and FIG. 8D show the prediction performance of CSB-BFRM on FDA approved drugs and clinical trial drugs;

FIG. 9 illustrates OTEs and off-targets of raloxifene, tamoxifen, paclitaxel, and fulvestrant on the cell cycle G1/S checkpoint and P53 signaling pathways. The right side is for the signal cascade in the cell cycle G1/S checkpoint signaling pathway while the left side is for the P53 signaling pathway. The drug-dose pairs are listed in the middle. The drug-targeted pathway was generated using IPA software (Ingenuity Systems, Inc., Redwood City, Calif., USA);

FIG. 10A, FIG. 10B, FIG. 10C, and FIG. 10D show the dose-response curves for raloxifene (FIG. 10A), tamoxifen (FIG. 10B), fulvestrant (FIG. 10C), and paclitaxel (FIG. 10D). Values of data points between 0% and 100% indicated that the drug inhibited cell growth. A growth percentage of −100 meant that all cells were killed. The dose-response curves for raloxifene (Evista®, Eli Lilly and Co., Indianapolis, Ind., USA) and tamoxifen implied that they induced cell death at higher dosages, while the curve for paclitaxel (Taxol®, Bristol-Myers Squibb, New York, N.Y., USA) showed that some experiments also cause the cell death. In contrast, the curve for fulvestrant (Faslodex®, AstraZeneca, plc; London, United Kingdom) indicateD that, at higher dosages, fulvestrant did not induce the cell death. The data source used was that of the Developmental Therapeutics Program (DTP) from the National Institutes of Health, National Cancer Institute (Bethesda, Md., USA);

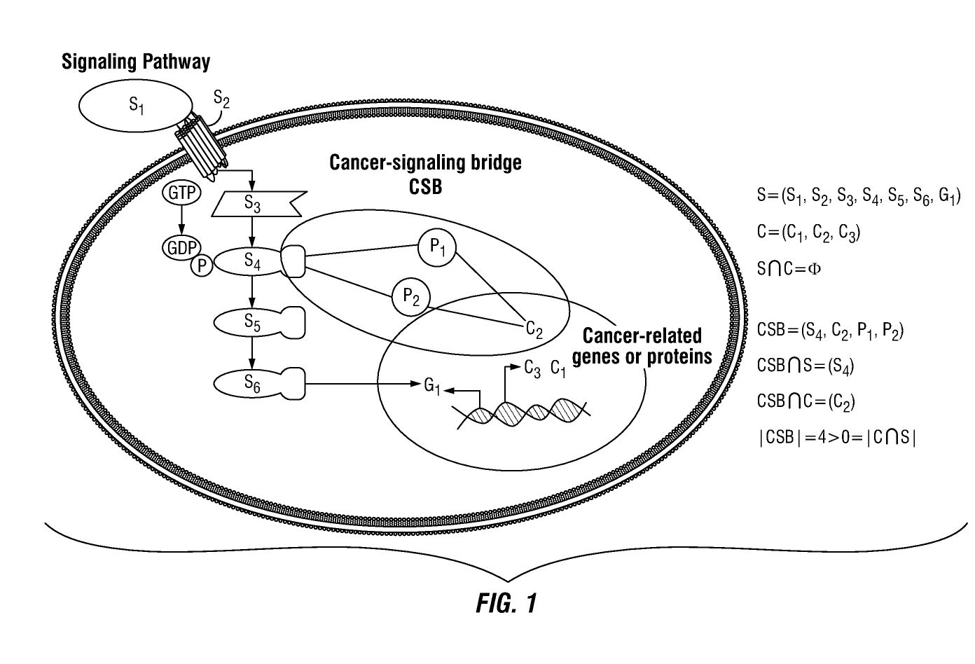

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Cancer involves a complex communication process, in which signals delivered through a pathway trigger an abnormal expression of cancer-related genes or proteins. The perturbed cancer-related genes or proteins may in turn generate positive feedback to the original signaling pathway and enhance cancer progression (Marks et al., 2009). One well-studied example of positive feedback in tumors is seen with transforming growth factor alpha (TGFα), a mitogenic wound hormone, which activates the RAS-RAF-MEK-ERK signaling pathway to enhance the effect of TGFα on the MAP kinase cascade. In turn, the RAS-RAF-MEK-ERK signaling pathway leads to increased production of TGFα, resulting in a positive feedback loop.

Many methods have been attempted to understand the communication between a known signaling pathway and certain disease-related genes or proteins, which may or may not be included in the signaling pathway. Well-characterized signaling pathways can take advantage of mutational analyses to identify important cancer related genes (Bentires-Alj et al., 2006). For example, in the RAS signaling pathway, mutational analysis of, PTPN11, led to the discovery that it encodes SHP2, which is required for full RAS activation in about one-half of Noonam and all of LEOPARD syndrome cases (Tartaglia et al., 2005). However, these mutational analyses are specific to the signaling pathways under investigation, and lack the capacity to identify large numbers of cancer-related genes. On the other hand, when little is known about the signaling pathway, computational methodologies can be deployed to obtain gene signatures to better understand the disease mechanism. For instance, breast cancer brain metastasis (Bos et al., 2009) and the development of tumor initiating cells (TICs) (Creighton et al., 2009). While these computational methods can detect almost all the differentially expressed genes, they often miss the underlying signaling mechanisms (Bos et al., 2009; Creighton et al., 2009).

Signaling pathways do not operate alone, but are connected together by proteins or protein modules (Marks et al., 2009). In the course of evolution, the protein modules were recombined into new patterns by genetic mutations. Innovations in signaling processing are brought about by unique combinations of existing building blocks, known as network motifs, rather than by invention of entirely new protein modules (Jin et al., 2007; Milo et al., 2002; Shen-Orr et al., 2002). Network motifs permit the study of not only signals between signaling pathways, but also the communication between signaling pathways and disease-related genes or proteins.

Thus, the proposed drug-repositioning platform consists of CSB model, bioinformatics software, and drug information databases. Among the twenty-one drug candidates identified in this study, three drugs, i.e., sunitinib (Sutent®, Pfizer Oncology, New York, N.Y., USA), dasatinib (Sprycel®, Bristol-Myers Squibb, Princeton, N.J., USA) and chloroquine (Aralen®, sanofi-aventis, US, LLC, Bridgewater, N.J., USA) were further analyzed for their in vitro and in vivo effects on human breast cancer TICs, and in murine xenograft models. These results showed that all three drugs had anti-TICs effects on at least two triple-negative cancer cell lines and anti-cancer effects in animal models (as demonstrated by the mammosphere formation efficiency assay, and the dilute transplantation assay, respectively). As a result of this finding, dasatinib is now currently in Phase II clinical trials for advanced breast cancer to assess the efficacy of this drug for inhibiting TICs.

### Development of a Cancer-Signaling Bridge-Model for Drug Repositioning

Understanding the signaling mechanisms of breast TICs is important for designing efficient therapeutics, and managing treatment strategies for breast cancer. The present invention provides a novel, network-based signature, and a comprehensive signaling map for identification of candidates for drug repositioning and, in particular, for therapy of breast TICs. The network-based signature described herein was based on an extended concept of network motifs, known as cancer-signaling bridges (CSBs), which can now be used to expand the cancer drug-targets of known signaling pathways. Using the profiles of TICs derived from CD44⁺/CD24^{-/low} breast cancer cells and mammospheres (MS) cells, network-based signatures were established. Facilitated by the signaling pathways that were highly connected with CSBs (e.g., MAPK, NOTCH, ECM-receptor, Jak-STAT, and Wnt), the high-confidence signaling paths automatically chosen out of the CSBs were identified and characterized using two scoring systems: Differential Expression Score (DES) and Signaling Pathway Score (SPS). These high-confidence signaling pathways were then used to construct a network-based signature that was characteristic for breast TICs.

In the definition of CSBs, -protein interaction network and signaling pathways were considered, as well as cancer-related genes or proteins. The data sources of protein-protein interaction networks, signaling pathways, and cancer-related genes or proteins are shown in Table 1:

TABLE 1
DATA SOURCES FOR DEFINING CSBS

| Data | Data Sources |
|---|---|
| Protein:protein interaction | IntAct, DIP, MINT, MIPS, BioGrid |
| networks | |
| Signaling Pathways | NCI-PID, BioCarta, KEGG |
| Cancer-related genes or proteins | OMIM |

The network elements in protein-protein interaction networks, called as network motifs, were used to define the CSBs, which could be identified using commercially available software (e.g., “mfinder” network Motifs Detection Tool, developed by the group of Uri Alon). In the protein-protein interaction network combined from multiple databases, two interaction patterns: triangle (for three proteins with three interactions) and square (i.e., four proteins with four interactions), were found to be characteristic network motifs. The CSBs were defined as subgraphs with an interaction pattern of a triangle or a square that could expand the proteins in signaling pathways to individual cancer-related genes or proteins. Mathematically, denoting S as the protein set of a signaling pathway and C as the coding-protein set of cancer genes, and [symbol] as the set of subgraphs having an interaction pattern of a triangle or a square, Equation 1 provides that [symbol] is a set of CSBs, which is a subset of [symbol] , and each CSB, of [symbol] (j=1,2, . . . , [symbol] ) satisfies that

|CSBⱼ∩S|>0,|CSBⱼ∩C|>0, and |CSBⱼ|>|CSB^{j}∩(S∩C)| (1)

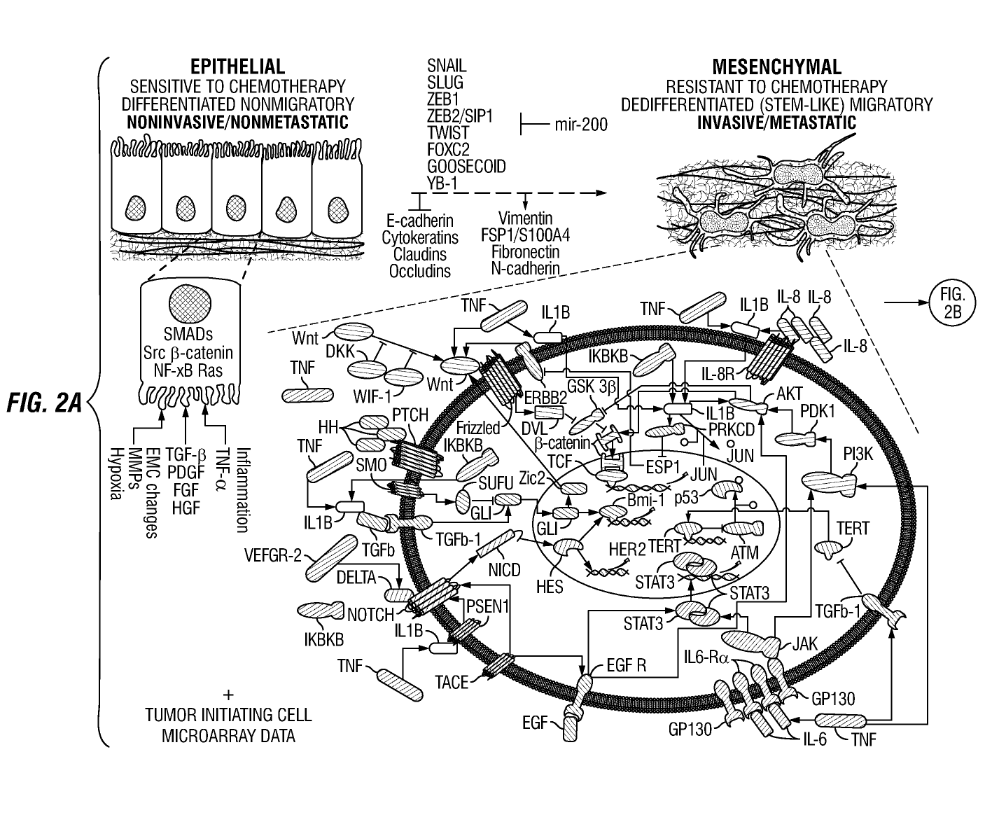

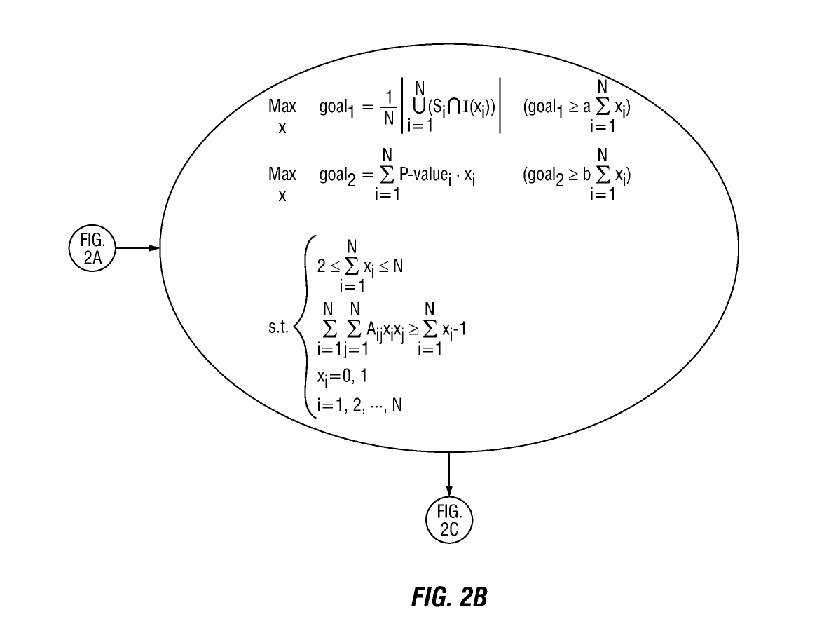

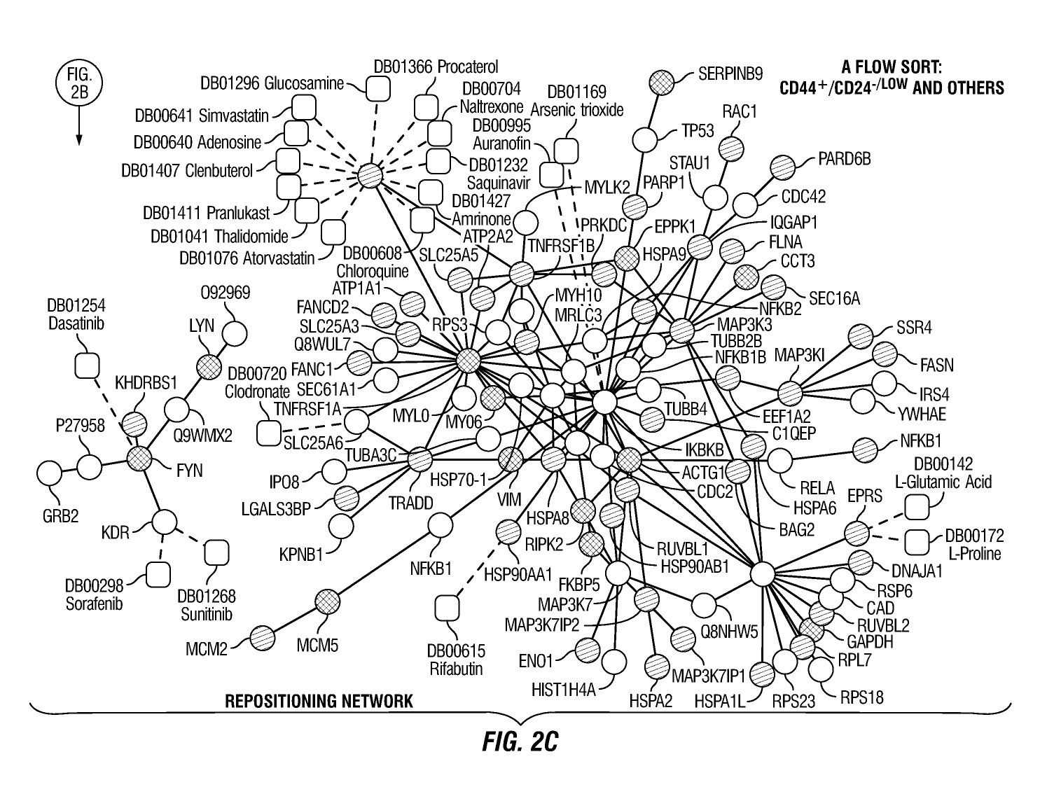

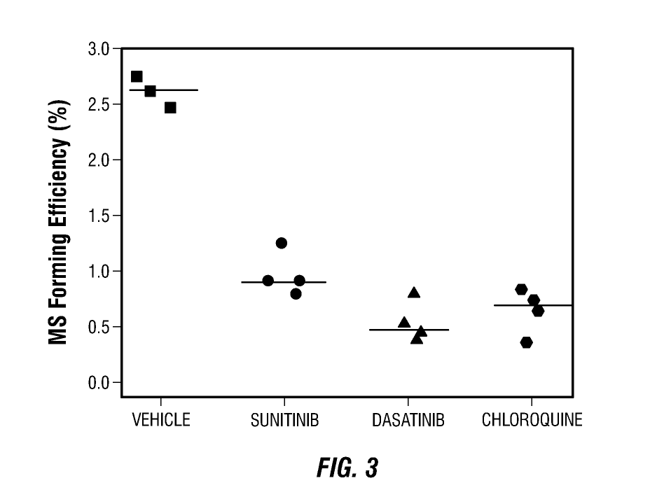

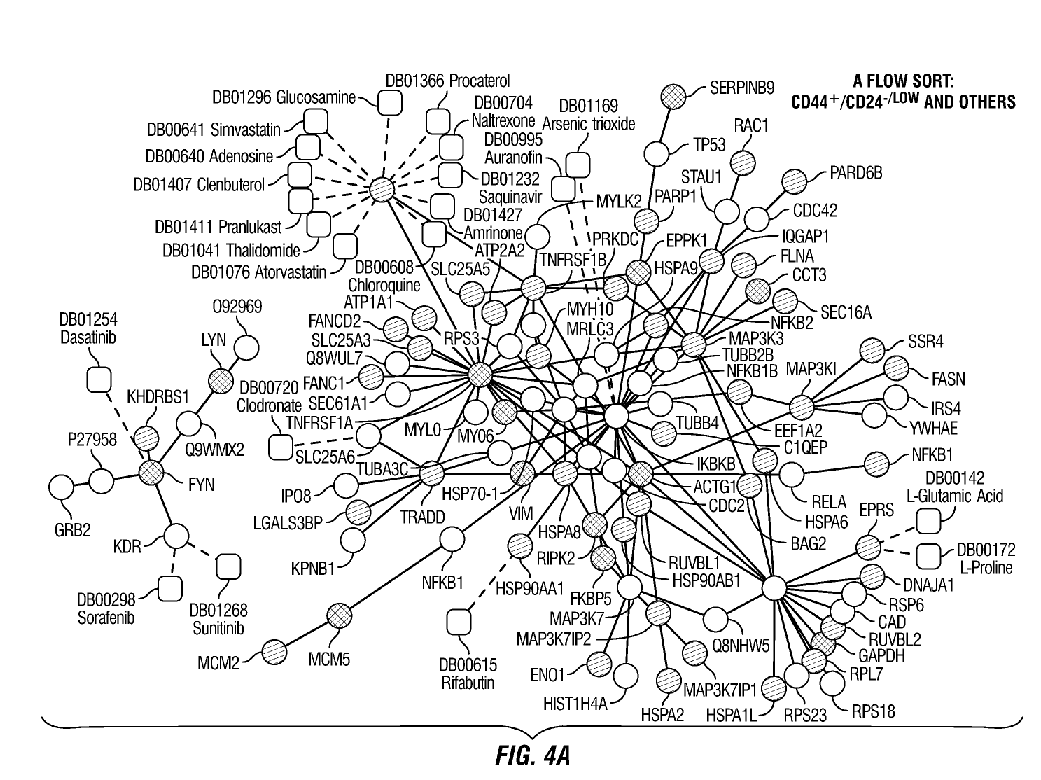

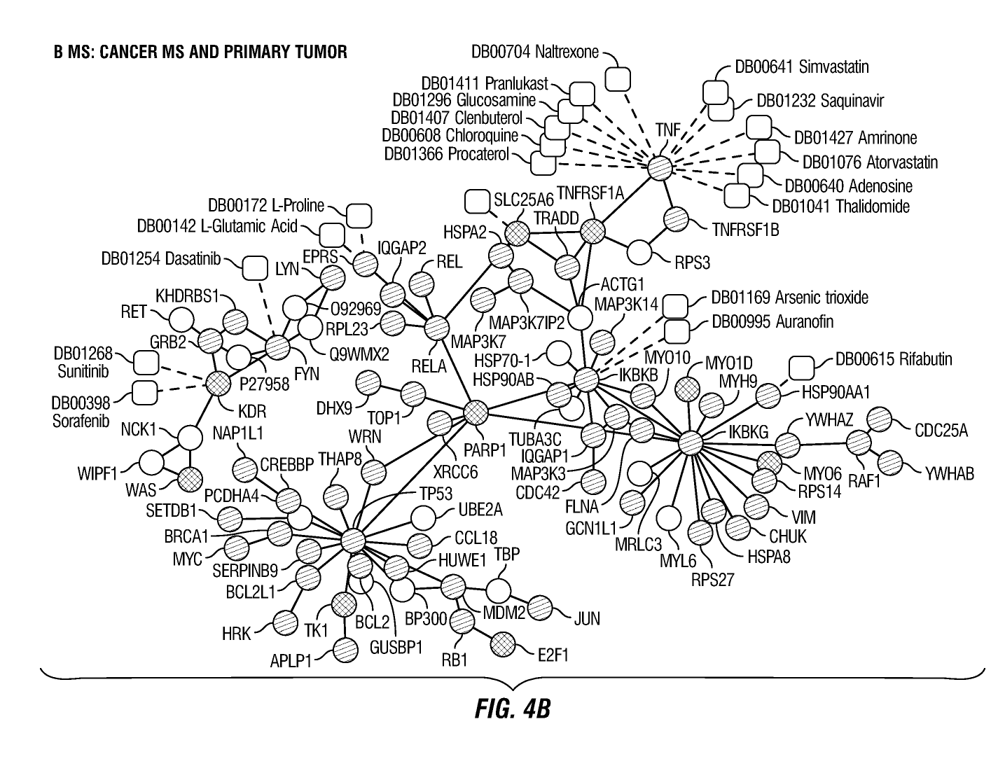

The identified CSBs facilitated the creation of a network-based drug repositioning signature (NS). Facilitated by CSBs, a CSB model has been proposed to integrate the signaling pathways and TIC expression profile into drug repositioning. In the CSB model, two types of scores were defined. First, a Signaling Pathway Score (SPS) was used to evaluate to what degree the considered NS is essential for the signal transduction of signaling pathways, which was defined as:

$${SPS}_{k} = \frac{1}{\vert {N}_{k} \vert} \vert {{⋃}_{l = 1}}^{\vert {N}_{k} \vert} {ζ}_{l} \vert ,$$

where Nₖ is a candidate NS, ζₗ is the subset of enriched signaling pathways. A second score, the Differential Expression Score (DES), was used to evaluate the association between the NS and TICs, which was defined as:

$${DES}_{k} = \frac{1}{\vert {N}_{k} \vert} {\sum }_{l = 1}^{\vert {N}_{k} \vert} - {log}_{10} ( P - {value}_{l} ) ,$$

where P-valueₗ is the P value of protein Pₗ of NS in the TIC gene expression profiles. The CSB mode, which is summarized in the flow chart shown in FIG. 2A, FIG. 2B, and FIG. 2C, aimed to maximize the two types of scores.

The CSB model could be formulated as an optimization problem, wherein:

$${Max}_{x} {goal}_{1} = \frac{1}{N} \vert {{⋃}_{i = 1}}^{N} ( {S}_{i} ⋂ I ( {x}_{i} ) ) \vert ( {goal}_{1} \geq a {\sum }_{i = 1}^{N} {x}_{i} )$$

$${Max}_{x} {goal}_{2} = {\sum }_{i = 1}^{N} P - {value}_{i} \cdot {x}_{i} ( {goal}_{2} \geq b {\sum }_{i = 1}^{N} {x}_{i} )$$

$$s . t . \{ \begin{matrix}2 \leq {\sum }_{i = 1}^{N} {x}_{i} \leq N \\ {\sum }_{i = 1}^{N} {\sum }_{j = 1}^{N} {A}_{ij} {x}_{i} {x}_{j} \geq {\sum }_{i = 1}^{N} {x}_{i} - 1 \\ {x}_{i} = 0 , 1 \\ i = 1 , 2 , … , N\end{matrix}$$

Using this relationship, the inventors applied the CSB model to the gene expression profiles of TICs derived from CD44⁺/CD24^{-/low} breast cancer cells and mammosphere (MS) cells to establish a network-based drug-repositioning signature. The resulting network-based drug-repositioning signature for CD44⁺/CD24^{-/low} cells was composed of 140 proteins and 132 protein-protein interactions, while the network-based drug-repositioning signature for mammospheres was composed of 153 proteins and 119 protein-protein interactions. Of nearly 2,000 FDA-approved drugs screened, 21 of them had targets that were contained in the network-based signatures of both CD44⁺/CD24^{-/low} and mammosphere cells, and were repositioned to TICs. Based on the methods described herein, these 21 drugs (identified in Table 2) were then selected as repositioned drug candidates for breast TICs.

TABLE 2
EXEMPLARY REPOSITIONED DRUGS IDENTIFIED BY
THE PRESENT INVENTION

| Drug | Target in the NS |
|---|---|
| DB00142 L-Glutamic Acid | EPRS |
| DB00172 L-Proline | EPRS |
| DB00398 Sorafenib | KDR |
| DB00608 Chloroquine | TNF |
| DB00615 Rifabutin | HSP90AA1 |
| DB00640 Adenosine | TNF |
| DB00641 Simvastatin | TNF |
| DB00704 Naltrexone | TNF |
| DB00720 Clodronate | SLC25A6 |
| DB00995 Auranofin | IKBKB |
| DB01041 Thalidomide | TNF |
| DB01076 Atorvastatin | TNF |
| DB01169 Arsenic trioxide | IKBKB |
| DB01232 Saquinavir | TNF |
| DB01254 Dasatinib | FYN |
| DB01268 Sunitinib | KDR |
| DB01296 Glucosamine | TNF |
| DB01366 Procaterol | TNF |
| DB01407 Clenbuterol | TNF |
| DB01411 Pranlukast | TNF |
| DB01427 Amrinone | TNF |

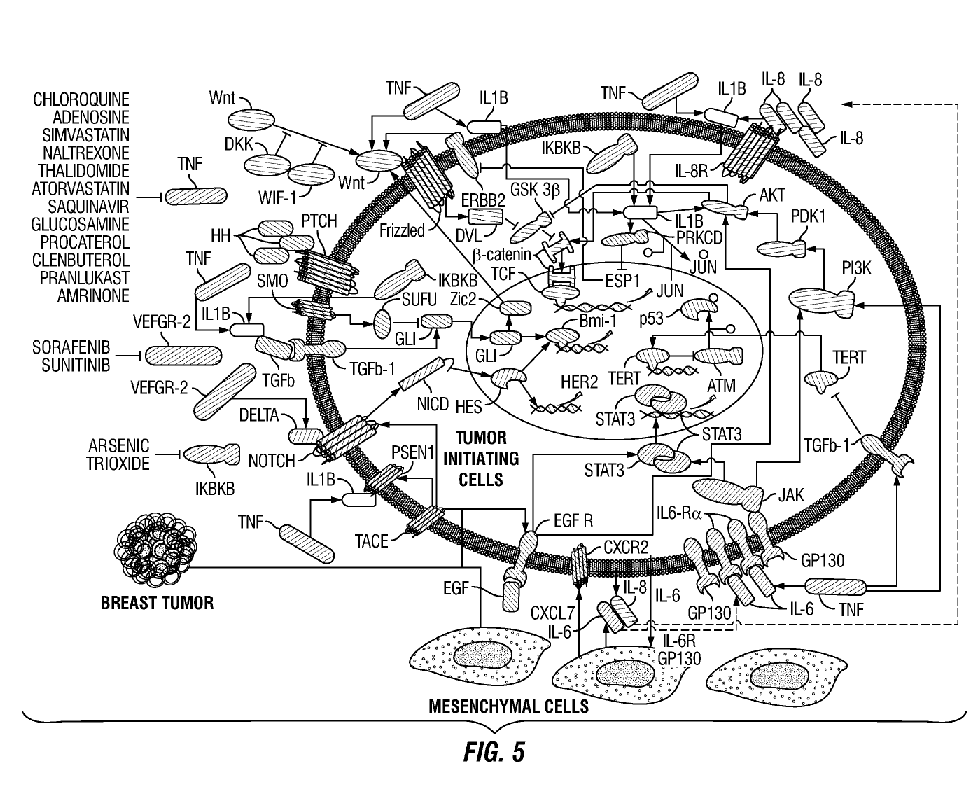

Using this mapping approach, the drugs targeting on TNF, KDR, and IKBKB, such as Sunitinib, Arsenic trioxide, and Atorvastatin, are repositioned for treating breast TICs, while the crosstalk between pathways such as Notch+hedgehog and Hedgehog+wnt+PI3K were used to identify drug combination candidates from the target combinations, TNF+KDR, and TNF+IKBKB (FIG. 5).

Development of DrugR Software Tool for Customized Repositioning of Drug Candidates

To facilitate ready adoption of the methods described herein, the inventors have also developed a software tool, named “DrugR,” for executing the computational methods described herein.

Developing a DrugMap Central Database for Analyzing Drug Repositioning Candidates

In addition, another drug database, termed “DMC” (or ‘DrugMap Central database’), was constructed to accurately collect and organize the information of drugs, especially information regarding their approval for use by the United States Food and Drug Administration (USFDA).

### Exemplary Definitions

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range. The term “e.g.,” as used herein, is used merely by way of example, without limitation intended, and should not be construed as referring only those items explicitly enumerated in the specification. In accordance with long standing patent law convention, the words “a” and “an” when used in this application, including the claims, denotes “one or more.”

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure, appreciate that many changes can be made in the specific embodiments that are disclosed, and still obtain a like or similar result without departing from the spirit and scope of the invention.

### Example 1

### A Systems Method for Drug Repositioning for Breast Cancer

This example describes a new network-motif based method to study the communication process between signaling pathways and individual cancer-related genes or proteins in order to expand cancer drug-targets of signaling pathways. A particular type of instances of network motifs, termed “cancer-signaling bridges” (CSBs), was identified in the present methods, which was shown to be enriched in the connections between oncogenic signaling pathways and cancer-related genes or proteins. These CSBs were used to expand the signaling pathways to different types of cancers, and we found that most CSBs are not shared by multiple types of cancers, but specifically connected to one type of cancer. Both drug-target and drug-effect analyses were performed on CSBs. It was found that the expanded signaling proteins are more likely to be targeted by anti-cancer drugs, and they are responsible for expanding the drug-effects from the targets in the signaling pathways to downstream cancer-related genes or proteins. The hypothesis generated by these drug analyses was that CSBs bring out the previous ignored pathways for targets therapy by transmitting the drug effect to the linked cancer-related genes or proteins.

To prove the hypothesis, the CSBs were applied to study drug repositioning in cancer. A Bayesian Factor Regression Model (based on the identified CSBs) was performed to reposition forty-nine anti-cancer drugs to ten different types of cancers. In the computational modeling, a repositioning score was proposed to identify the effectiveness of a particular known anti-cancer drug on a specific type of cancer, and this repositioning signature was used to illustrate why the anti-cancer drug was effective on each particular type of cancer. The predicted indications obtained from the repositioning scores were validated using information about FDA approval status, clinical trials stages, and also in “wet-lab” experiments on cancer cell proliferation.

Importantly, the CSB-based repositioning methods described herein could accurately predict more than 80% of the indications currently approved by the FDA, and almost 70% of the indications that are currently in Phase II or Phase III clinical trials (Lamb et al., 2006; Lamb et al., 2003). Furthermore, a total of fifteen repositioning predictions for three targeted-therapy anti-cancer drugs were validated in five different types of cancers by “wet-lab” studies, indicating that at least 87% (13 indications) of the repositioning predictions were correct.

Materials and Methods

Cell Lines and Reagents

Five tumor cell lines were used in these studies: HT-29, human colorectal adenocarcinoma; H69, human small cell lung cancer (SCLC); H226, human non-small cell lung cancer (NSCLC) (squamous carcinoma); LNCaP, human prostate carcinoma; and U87-luc glioblastoma cell line. H69, H226, and LNCaP cells were grown in American Type Culture Collection (ATCC)-formulated RPMI-1640 Medium (ATCC, cat#30-2001; Bethesda, Md., USA) supplemented with 10% fetal bovine serum (FBS) and 1% penicillin/streptomycin (Invitrogen, Carlsbad, Calif., USA). HT-29 cells were cultured in ATCC-formulated McCoy's 5a Medium (Modified) (ATCC, cat#30-2007). U87-luc cells were grown in Dulbecco's Modified Eagle Medium DMEM (Invitrogen/GIBCO, cat#11995) containing 10% FBS, 1% penicillin/streptomycin, 1% L-glutamine, and 0.5 mg/mL antibiotic G-418 sulfate (Sigma-Aldrich, cat# G5013-1G Sigma-Aldrich, St. Louis, Mo., USA. All cell cultures were incubated at 37° C. in a humidified, 5% CO₂ atmosphere, and were in logarithmic growth phase at the initiation of the studies.

Three targeted-therapy drugs (gefitinib, paclitaxel, and flutamide) were used in this study. Gefitinib was purchased from LC Laboratories (Woburn, Mass., USA) and stored at −20° C. Flutamide and paclitaxel (10 mM in dimethyl sulfoxide [DMSO]) were both obtained from Sigma-Aldrich (cat#, F9397-1G, and Library of Pharmacologically Active Compound #1280, LOPAC1280, respectively), and stored at −20° C. All three compounds were diluted in cell culture medium prior to use, with the final DMSO concentration in all studies less than 0.1%.

Cell Proliferation Assay

Cell proliferation was analyzed using CellTiter 96 AQueous One Solution Cell Proliferation Assay (Promega, Madison, Wis., USA) according to the manufacturer's instructions. Briefly, 5,000 cells of each cell line in 100 μL cell culture media were seeded in each well of 96-well white wall/clear bottom plates in duplicate and incubated overnight. On the next day, cells culture media were replaced with media containing either compound for the treatment. The individual compounds and concentrations used in this study were: gefitinib (1 μM, 20 μM) 1311, paclitaxel (0.1 μM, 1 μM) (Terzis et al., 1997), and flutamide (0.1 μM, 2 μM) (Zhigang et al., 2008). After 48 hours of treatment, CellTiter™ 96 Aqueous One-Solution Reagent was added to each well of a 96-well plate. After 3-hrs' incubation, absorbance at 490 nm was measured using the FLUOstar™ Omega Microplate Reader (BMG LABTECH, Cary, N.C., USA). Background absorbance (490 nm) was corrected by subtracting the average 490 nm absorbance of the “no cell” control wells from absorbance values obtained for each of the test wells. The proliferative ratio (% surviving cells) in each sample was calculated by dividing the corrected absorbance value of treated cells by that of control cells with no drug treatment. The results were expressed as mean with standard errors from two independent studies.

Results and Discussion

Mapping Between Known Signaling Pathways and Cancer-Related Genes or Proteins

If cancer-related genes or proteins can be directly mapped to the signaling pathway of interest, it would help explain how oncogenic signals are transmitted throughout the signaling pathway. Such a direct mapping, however, can only cover a small fraction of cancer-related genes or proteins. The inventors investigated the overlaps between cancer-related genes and gene-coding proteins in Online Mendelian Inheritance in Man (OMIM) (Hamosh et al., 2005; Yildirim et al., 2007), and the signaling proteins in Nature Curated pathways (NCI-PID) and BioCarta pathways (Schaefer et al., 2009). Only a small number of cancer-related genes or proteins can be found in the existing signaling pathways, and more than thirty percent of signaling pathways do not contain any cancer-related genes or proteins. The result indicated that most cancer-related genes or proteins could not be found in, or mapped to, the known signaling pathways.

CSBs (Cancer-Signaling Bridges)

Network motifs were identified by a high-throughput physical protein-protein interaction (FPI) network. An enrichment analysis was applied to the instances of network motifs. In the statistical analysis, CSBs were defined as the specific instances of network motifs that enable signaling pathways to connect with cancer-related genes or proteins. CSB mapping allows signaling pathways to connect with significantly more cancer-related genes or proteins compared to database mapping (P<10⁻¹⁰, Mann—Whitney U test). To avoid bias, a randomized process was designed to evaluate the enrichment of CSBs in the connection between signaling pathways and cancer-related genes or proteins. The results indicated that CSBs were significantly enriched in most connections between oncogenic signaling pathways and cancer-related genes or proteins. Additionally, it was found that CSBs could be categorized according to particular cancer type. It was also shown that CSBs connected with the ln3K/AKT signaling pathway. It was also shown that distinct CSBs were responsible for expanding the PI3K/AKT signaling pathway to different types of cancers. These results demonstrated that most CSBs were not shared by multiple types of cancers, but that they were useful in expanding the information of signaling pathways to different types of cancers.

Drug-Target and Drug Effect Analyses on CSBs

Targeting signaling pathways is an important strategy for generating therapeutic effects from anti-cancer drugs (Ande et al., 2009; Knauf et al., 2009; Lu et al., 2009; Yarde et al., 2009). Of interest was identifying the function of CSBs during drug treatment. Therefore, both drug-target and drug-effect analyses were performed on the resulting CSBs.

The process of “drug-target analysis” (DTA) investigates which genes or proteins in signaling pathways, and which cancer-related genes or proteins in OMIM^{cancer} were more likely to be targeted by anti-cancer drugs. A quantitative criterion, known as target-rate, was defined to evaluate to what extent a set of genes or proteins was targeted by an anti-cancer drug. This target-rate was calculated as the ratio between the number of anti-cancer drugs whose targets were included in the set, and the number of genes or proteins present in the set. The inventors demonstrated the distribution of anti-cancer drugs in signaling pathways, CSBs, and OMIM^{cancer}, as well as the overlap between the three sets, where OMIM^{cancer} denotes the genes or proteins related to cancer in the OMIM^{cancer} set. It was found that the genes or proteins in both signaling pathways and OMIM^{cancer} that overlapped with CSBs were significantly more likely to be targeted by anti-cancer drugs (P<10⁻⁵, Fisher's exact test).

Excluding the genes or proteins in the connection with CSBs, only a few genes or proteins in OMIM^{cancer} were targeted by anti-cancer drugs. The 394 cancer-related genes or proteins were targeted by only three known anti-cancer drugs. On the other hand, excluding the genes or proteins in CSBs overlapped with signaling pathways and OMIM^{cancer}, the remaining targets were significantly less likely to be targeted by anti-cancer drugs (P<10⁻¹⁴, Fisher's exact test). The 1,305 proteins were targeted by only 31 anti-cancer drugs.

The process of “drug-effect analysis” (DEA) examined to what degrees the genes or proteins of signaling pathways, CSBs, and OMIM^{cancer} were perturbed by the effects that were generated by anti-cancer drugs. The quantitative criterion, E-score, was proposed to evaluate the drug effects on a gene set or a protein set of interest. E-score was computed by a non-parameter statistical test, the Kolmogorov-Smimov test (K-S test), that examined whether two underlying one-dimensional probability distributions differ. Calculating the E-score of a particular anti-cancer drug required the expression profiles from human cancer cells cultured under two conditions, treatment vs. control, in which the former refers to the experiments to treat the cells by the anti-cancer drug while the latter included those without any treatment. The resulting expression profiles were used to evaluate the particular drug effects by considering the fold-changes of the genes or proteins that were defined as the ratio of corresponding treatment-to-control values. The fold-change for two sets of genes or proteins was then used to define E-score, that is, the set of the genes or proteins of interest compared to the whole set of genes in the expression profiles. By comparing the two distributions of fold-changes, the resulting E-score quantitatively evaluated the drug effects on the particular gene- or protein-set of interest.

The resulting E-score analysis was applied to the expression profiles of fifty-six anticancer drugs deposited in the connectivity map O₂ (Lamb et al., 2006; Lamb et al., 2003). To compare the drug effects on the signaling pathways, CSBs, and OMIM^{cancer}., the corresponding three types of E-scores for all fifty-six anti-cancer drugs were calculated. It was found that the E-scores for OMIM^{cancer} were significantly higher than those for signaling pathways and CSBs (P<10⁻²⁰, Mann—Whitney U test), which meant that the anti-cancer drugs generated relatively high effects on cancer-related genes or proteins. These results also indicated that the drug-effects could be transmitted from the drug-targets in signaling pathways to other cancer-related genes or proteins.

Applying CSBs to Drug Repositioning

To approve the hypothesis generated by drug analyses, CSBs were utilized in a novel method to perform drug repositioning on anti-cancer drugs. A new type of statistical factor analysis has been developed based on the CSB concept to reposition anti-cancer drugs. Using a well-known statistical factor analysis, the Bayesian Factor Regression Model (BFRM), for drug repositioning (Andrechek et al., 2008; Andrechek et al., 2009; Chang et al., 2009; Bild et al., 2006; Ashburn et al., 2004) permitted the recognition of downstream anti-cancer drug effects on specific types of cancer, thus facilitating the repositioning or repurposing of “old” or “known” drugs.

A “repositioning score” (RS) was proposed in the analysis to rank the effectiveness of repositioning, and it was used to reposition 49 known anti-cancer drugs to ten different types of cancers. The 49 anti-cancer drugs are those used in drug-effect analysis whose targets include at least one protein. For each cancer, the CSBs connected with the genes or proteins related to the cancer in the OMIM^{cancer} were first identified, then, BFRM was performed on an input matrix, which was composed of the expression profiles of the identified CSBs treated with the 49 anti-cancer drugs. BFRM deconvolutes the expression changes or fold-changes defined by the gene expressions of the genes or proteins in the identified CSBs for conditions of before and after drug treatments into the values of a weight matrix and those of a score matrix.

For each anti-cancer drug, the RS for the cancer of interest was determined by the analytical outputs of BFRM on the particular cancer. The targets of the anti-cancer drug were the key to defining the RS. The RS was defined as the maximal weighted score across the targets, and each weighted score is determined by the weights of the target across the signatures in the weight matrix and the scores of the anti-cancer drug across the signatures in the score matrix.

Since the RS was determined by the weights of the targets in the weight matrix, it needed at least one target for each drug to be included in the identified CSBs. However, not all of the drugs could find targets in the identified CSBs. Accordingly, the 49 anti-cancer drugs were classified into four separate classes: Bridge Drugs, Neighbor-Bridge Drugs, Neighbor-PPI Drugs, and Not-Associated Drugs.

Bridge drugs are the drugs that directly target the identified CSBs while neighbor-bridge drugs and neighbor-PPI drugs are those drugs whose targets indirectly reach identified CSBs if they are assisted by at least another bridge and one PPI. For the later two classes, the expanded proteins in the identified CSBs were called as the expanded targets of the drugs. The last class was termed “not-associated” because their targets could not be connected with any of the identified CSBs. Bridge drugs can easily find their targets in the identified CSBs and obtain repositioning scores using the proposed repositioning method. Neighbor bridge drugs and neighbor PPI drugs find their expanded targets in the identified CSBs and obtain the repositioning scores by the same method. Not-associated drugs could find neither targets nor expanded targets in the identified CSBs, which indicated that these drugs may have little association with the particular cancer. They were assigned relatively poor repositioning scores by selecting a random value from −1 to 0.

The RS characterizes the effects of the repositioned drug on the identified CSBs of the cancer of interest. A positive score indicates that the repositioned drug is able to inhibit the cancer. Otherwise, a negative score (or lower than zero) indicates the drug generates a reverse effect on the cancer (no positive effect for inhibiting the cancer cells, or accelerating cells to grow).

Validation of the Repositioning Results

Some disease treatment indications found in this repositioning study have already been approved by the FDA, while others are presently undergoing Phase II or Phase III clinical trials. These indications were first validated by the available information from FDA and from the results of published clinical trials. It was found that more than 80% of FDA approved indications (and nearly 70% of indications in the Phase II or Phase III clinical trials) could be predicted by positive RS's.

At the same time, studies on cell proliferation were also performed to validate the predicted indications. The experiments were implemented on three targeted-therapy drugs, i.e., gefitinib, paclitaxel, and flutamide, across five cancer cell lines, i.e., brain tumor, prostate cancer, non-small cell lung cancer, small cell lung cancer, and colorectal cancer. A total of fifteen indications were validated, of which ten indications were predicted by positive RS's, and five indications were predicted by negative RS's. The studies demonstrated that all ten of the positive indications showed a significant decrease in proliferation rates, and three of the five negative indications demonstrated an increase in proliferation rates. The prediction accuracy was the ratio between validated predicted indications and all of the predicted indications (i.e., 13/15 or 87%).

The results for FDA approval status, clinical trial information, and wet-lab experiments validation indicate that the CSB-based drug repositioning method facilitates repositioning drugs for cancer purpose.

Understanding the Repositioning Mechanisms by CSBs

The question remains as to how the repositioned drugs work on certain types of cancers, but not others. To better understand the mechanism of drug repositioning, the repositioning signature was used to investigate the signaling networks that are responsible for the effects of the repositioned drugs. The repositioning signatures were composed of the CSBs that connect the targets with the genes or proteins with the relatively high drug effects generated by the drugs. Moreover, these repositioning signatures are defined by the outputs of BFRM, i.e. weight matrix and score matrix. By comparing the weighted scores of targets, or expanded targets, the target with the highest score was first identified. The weights of the identified target were then used to select non-trivial signatures whose weights in the weight matrix were not equal to zero. Eventually, the genes or proteins with the highest non-trivial signatures effects were filtered out by the effect values defined by the weight matrix and score matrix. Here, the effect value on a gene or a protein in the repositioning signature has a little difference from the repositioning score of an indication. The former is derived from a specific signature of the identified target, and it is the highest value across the nontrivial signatures. The latter is an overall score by considering all the nontrivial signatures of the target.

As an illustrative example, the repositioning signature of gefitinib was used to illustrate why gefitinib has positive effects on non-small cell lung cancer and colorectal cancer.

The five repositioning signatures for the five approved indications of gefitinib were also determined. Gefitinib has a single target, epidermal growth factor receptor (EGFR), and has been approved for non-small cell lung cancer by the FDA. In these studies, it was demonstrated that gefitinib also has positive effects on the colorectal cancer cell line, HT-29.

The conventional interpretation on the mechanism of gefitinib is based on the functions of the target, EGFR. The study on EGFR has displayed that gefitinib inhibits the epidermal growth factor receptor (EGFR) tyrosine kinase by binding to the adenosine triphosphate (ATP)-binding site of the enzyme, and thus the function of the EGFR tyrosine kinase in activating the Ras signal transduction cascade is inhibited; and malignant cells are inhibited (Wishart et al., 2008).

However, the identified repositioning signatures facilitate a much more comprehensive understanding on the conventional interpretation on the mechanism of action of gefitinib. Because the CSBs were specific to different types of cancers, the repositioning signatures for different indications identified by CSBs were also distinct from each other. Facilitated by the repositioning signatures, it was revealed that the reason why gefitinib generates different effects on different types of cancers is that gefitinib is using distinct ways to impose their effects on the cancer-related genes or proteins. For the effective indications, non-small cell lung cancer and colorectal cancer, the repositioning signatures not only include the genes or proteins with relatively higher effects, such as, RALGDS and FHL2, but also involve in the key genes or proteins responsible for cell proliferation or growth, that is, PI3KCA and KRAS (non-small cell lung cancer), as well as CTNNB1, TP53, BCL2L1, KIN, and BCL2 (colorectal cancer). The key genes or proteins have been extensively validated by cell cultures or mouse models in the publish literatures.

Proliferation of NSCLC cells in mouse was increased by oncogenic K-RAS [KRAS2] protein (Konstantinidou et al., 2009). Interference of human PIK3CA mRNA by siRNA decreased growth of NCI-H460 cells (Wee et al., 2009). Interference of human P53 [TP53] gene by siRNA decreased proliferation of SW480 cells in cell culture (Yan et al., 2008). Interference of human BETA-CATEN1N [CTNNB1] gene by siRNA decreased proliferation of Caco-2 cells (Zhang et al., 2007). Human KIN17 [KIN] antisense [antisense RNA] [antisense RNA] decreased clonogenic growth of RKO cells (Biard et al., 2002). In mouse, human BCL2 human BCL-XL [BCL2L1] antisense DNA decreased growth of colorectal cancer cell lines (Gautschi et al., 2001). However, the repositioning signatures for small cell lung cancer, brain tumors, and prostate cancer showed relatively lower effects, and did not involve in any genes or proteins related to cell proliferation or growth.

Conclusion

In this example, a new method for expanding the cancer drug-targets of signaling pathways and applied it to reposition anti-cancer drugs has been presented. The new method employs a novel type of instances of network-motifs, CSBs, to expand the cancer drug-targets in signaling pathways to include individual cancer-related genes or proteins. The expanded signaling network enabled investigation of the effects of drugs more comprehensively and reliably than before. Using the methods described herein, BFRM repositioned 49 known cancer drugs to ten different types of cancer.

### Example 2

### Method for Transcriptional Response Analysis to Facilitate Drug Repositioning

In this study, the OTE-based method described above was further refined to repurpose drugs for cancer therapeutics, based on transcriptional responses made in cells before and after drug treatment. Specifically, the identified CSBs were integrated with a Bayesian Factor Regression Model (BFRM) to form a new hybrid method termed CSB-BFRM. Using breast and prostate cancer cells and in promyelocytic leukemia cells, the CSB-BFRM analysis was demonstrated to accurately predict clinical responses to >90% of FDA-approved drugs and >75% of experimental clinical drugs that were tested. Mechanistic investigation of OTEs for several high-ranking drug-dose pairs suggested repositioning opportunities for cancer therapy, based on the ability to enforce Rb-dependent repression of important E2F-dependent cell cycle genes. Together, these findings establish new methods to identify opportunities for drug repositioning or to elucidate the mechanisms of action of repositioned drugs.

This new method of off-target drug repositioning for cancer therapeutics is based on transcriptional response, and introduces prior knowledge of signaling pathways and cancer mechanisms into the off-target repositioning process. The use of CSBs to connect signaling proteins to cancer proteins whose coding genes have a close relationship with cancer genetic disorders has been demonstrated. By coupling CSBs analysis with a powerful statistical regression model (BFRM), the OTEs of drugs on signaling proteins can now be identified. This off-target repositioning method has thus been termed “CSB-BFRM.”

CSB-BFRM analysis was applied to three exemplary cancer transcriptional response profiles, and it was demonstrated that CSB-BFRM could accurately predict the activities of the FDA-approved drugs and clinical trial drugs for all three cancer types. Furthermore, the identified OTEs and off-targets were used to explain the action of the repositioned drugs. Four known drugs each with two different doses, or eight drug-dose pairs repositioned to MCF7 breast cancer cell line [raloxifene (0.1 μM and 7.8 μM), tamoxifen (1 μM and 7 μM), fulvestrant (1 μM and 0.01 μM), and paclitaxel (4.6 μM and 1 μM)] were investigated. It was shown that these drugs inhibited the transcription of certain key cell cycle genes by enhancing the Rb-dependent repression of E2F-mediated gene transcription. They exhibited negative OTEs on the off-targets, the heterodimer E2F and DP-1, and the kinases CDK2 and CDK4/6, of Rb, but positive OTEs on the inhibitors p15 and SCF of the Rb's kinases. The results were consistent with the dose-response curves derived from the Developmental Therapeutics Program (DTP) of the National Cancer Institute (NCI).

Overview of Main Model Equations

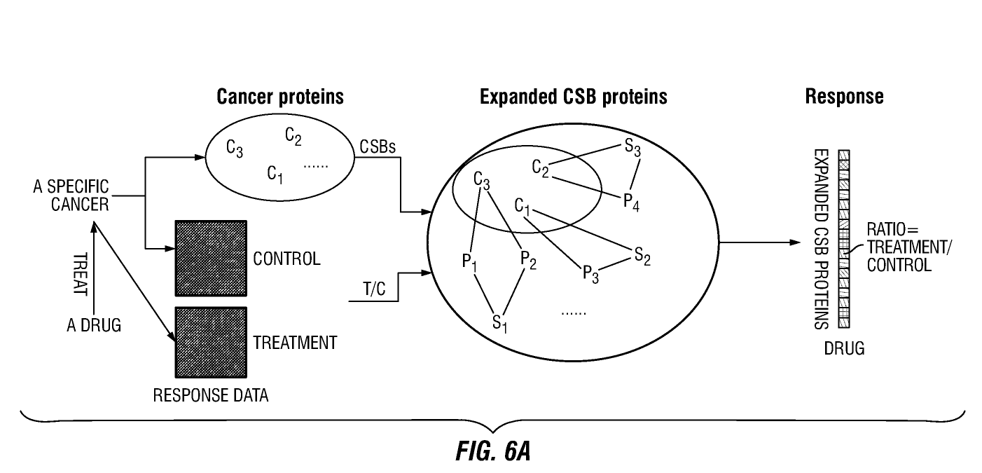

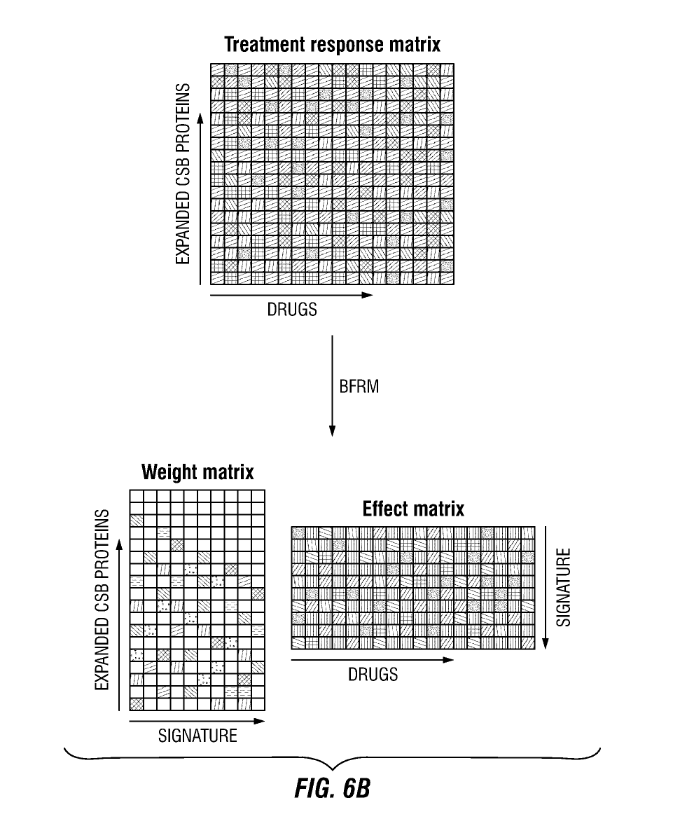

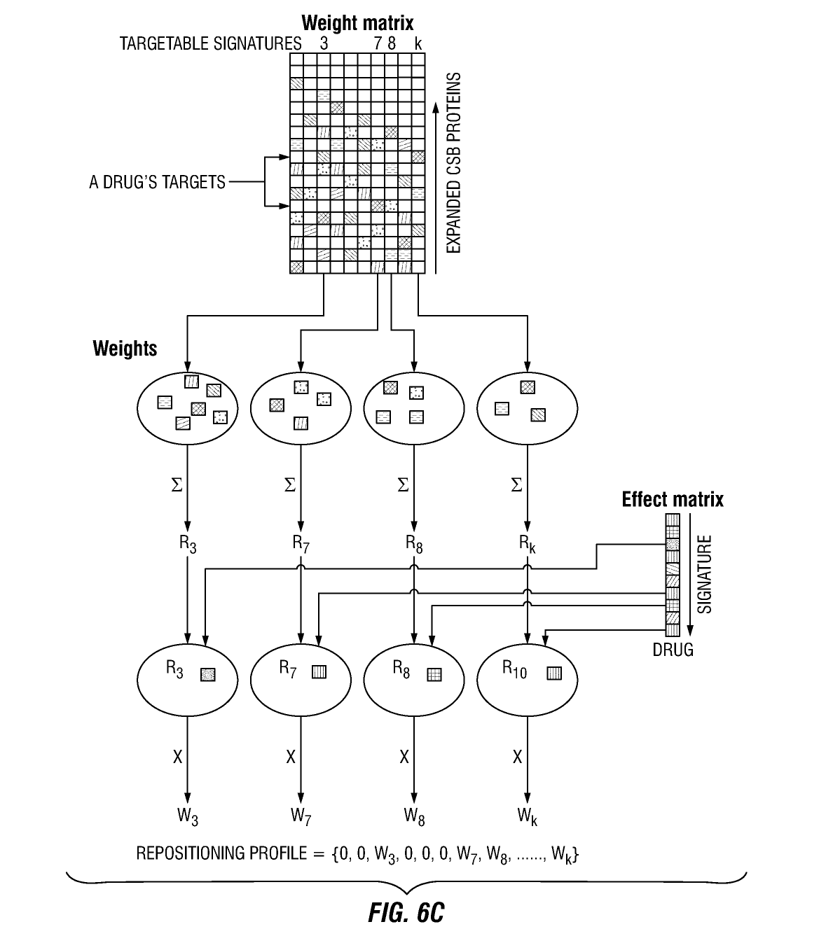

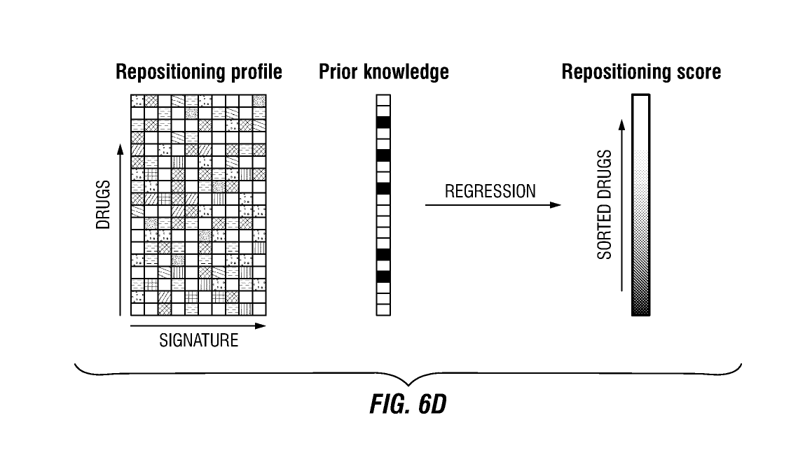

The strategy for off-target drug repositioning is illustrated in FIG. 6A, FIG. 6B, FIG. 6C, and FIG. 6D. Facilitated by CSBs, a new method was established to facilitate drug repositioning for complex diseases, and in particular, for improving therapies for human cancers.

Major Assumptions of the Model

CSB Definition

S is denoted as a protein set of a signaling pathway (i.e., NCI-PID (Pathway Interaction Database) or BioCarta pathway (Schaefer, 2009), C is denoted as a cancer protein set defined by the Online Mendelian Inheritance in Man (OMIM) database (Hamosh et al., 2005), in which each protein's coding gene (or genes) has a close relationship with a cancer genetic disorder, Π is the instance set of network motifs (Milo et al., 2002), Π^{S, C} is an subset of Π, where an instance comprises a set of proteins and a number of protein-protein interactions between them. Each CSB is a specific instance of one type of network motif; its protein set is denoted as CSBⱼ (jε{1,2, . . . , |Π^{S, C}|}) A CSB satisfies that,

|CSBⱼ∩S|>0,|CSBⱼ∩C|>0, and |CSBⱼ|>|CSBⱼ∩(S∩C) (A)

Off-Target Repositioning Method, CSB-BFRM

BFRM Model

Bayesian Factor and Regression Modeling (BFRM) (Bild et al., 2006; Chang et al., 2009) was applied to the off-target drug repositioning. BFRM deconvolutes the cancer transcriptional response data into signatures with a model of the form,

Xᵢ=Āλᵢ+φᵢ(i=1, 2, . . . , m) (B)

where X is an n dimension vector of fold-change (treatment vs. control) of drug i in the cancer transcriptional response data; X_{j,i}, j=1, 2, . . . , n, is the median value of fold-changes of gene j in consideration of corresponding instances treated by drug i; m is the number of drugs; and n is the number of the coding-genes for the CSB proteins expanded by the cancer proteins of a specific cancer type. Ā=(α₁,α₂, . . . , αₖ) is a sparse n×k matrix whose columns define the signatures Sₗ, l=1, 2, . . . , k, and each numerical value Ā_{j,i} defines the weight of gene j in the gene signature Sₗ. To address which parts of the cancer signals are responsible for the unknown pharmacological mechanisms and to what extent they are targeted, the CSB-BFRM method needs to identify signatures (the targeted parts in the cancer signals) and effects (OTEs on the targeted parts) (FIG. 6B). Thus, a weight matrix, A, was defined as a combination of one output of BFRM, Ā, and another matrix, P=(ρ₁,ρ₂, . . . , ρₖ), that contains the (sparse) probabilities that each gene is associated with each signature. The matrix, Λ=(λ₁,λ₂, . . . , λₘ), was termed an effect matrix. Each numerical value, Λ_{l,i}, defines the effect of drug i imposed on the gene signature, Sₗ·Ψ=(φ₁,φ₂, . . . , φₘ) reflects measurement error and residual biological noise.

Repositioning Profile

The OTEs of a drug on a specific cancer are defined as a repositioning profile using A and Λ (FIG. 6C). A repositioning profile, Ω=(ω₁,ω₂, . . . , ωₘ)^{T}, is an m×k matrix to characterize the overall effects of m drugs on k signatures. The known drug targets are essential for identification of a repositioning profile. The targetable signatures are defined by the non-zero weights at the rows of the targets across signatures of A. The targetable signatures for drug i was denoted as a set Tᵢ. For each targetable signature, tεTᵢ, the product was defined between Rₜ and the effect score Λ_{i,t} as the overall effect of drug i imposed on signature t, Ω_{i,t}=Rₜ×Λ_{i,t}, where

$${R}_{t} = {\sum }_{j = 1}^{n} {A}_{j , i}$$

denotes the response (or total weight) of the signature t to the drug i. The repositioning profile for drug i, ωᵢ, i=1, 2, . . . , m, is defined as,

$${ω}_{i} = \{ \begin{matrix}{R}_{t} \times {Λ}_{i , t} , & t ∈ {T}_{i} \\ 0 , & t ∉ {T}_{i}\end{matrix} \qquad ( C )$$

Because the target information for certain drugs may be unavailable, to define repositioning profiles for such drugs, a randomized process was used to simulate the targets of these drugs. To reduce the computation bias, the randomized process was repeated 1,000 times, and a sequence was degenerated for the repositioning profiles, Ξ=(Ω¹,Ω², . . . , Ω^{1,000}).

Repositioning Score

The identified repositioning profile was then applied to define a numerical value, termed the repositioning score, to distinguish the OTEs of the drugs. A supervised regression model, Support Vector Regression (SVR), was used to define the repositioning score. If a drug, i, is approved by the FDA or undergoing clinical trials, the element of the label vector for prior knowledge, Lᵢ, equals 1. SVR outputs a regression prediction vector, P^{h}, for each regression between repositioning profile Ω^{h}, h=1, 2, . . . , 1,000, and the label vector, L. P^{h} was sorted in descending order, and each drug's rank in the sorted P^{h} was recorded in a repositioning score vector, [symbol] . Thus, a sequence for repositioning score was defined:

Θ=( [symbol] , . . . , [symbol] ) (D)

The repositioning score for each drug was defined as mean±standard variation across the 1,000 repositioning score vectors.

Off-Targets and OTEs

The proposed repositioning score recognizes a drug's activity from the OTEs on the targetable signatures that comprise a number of off-targets. Off-targets were identified as those CSB proteins whose OTEs were non-zero. For a drug, i, its OTE on a CSB protein, j, j=1, 2, . . . , n, in a targetable signature t, tε{1, 2, . . . , k}, is defined as the product of A_{j,t} and Λ_{t,i}. Thus, the OTEs that drug ion the targetable signature t is a vector:

E_{i,t}=(A_{1,t}·Λ_{t,i},A_{2,t}·Λ_{t,i}, . . . , A_{m,t}·Λ_{t,i})^{T} (Ea),

and the OTE of drug i on CSB protein j was defined as the summation of all E_{i,t,j}(t=1, 2, . . . , k) across all of the targetable signatures, Tᵢ,

$${OTE}_{i , j} {\sum }_{t = 1}^{\vert {T}_{i} \vert} {E}_{i , t , j} \qquad ( Eb )$$

Materials and Methods

The drug-treated transcriptional response data were derived from Connectivity Map 02 (CMAP 02) (Lamb et al., 2006). There were 6,100 treatment instances, in which 6,066 instances were treated on 3 types of cancer cell lines: MCF7 breast cancer cell line, PC3 prostate cancer cell line, and HL60 promyelocytic leukemia cell line. Each instance has a treatment case for one drug with one dosage and variable numbers of controls (1, 5, or 6). There were 3,095, 1,742, and 1,229 instances designed for MCF7, PC3, and HL60 cell lines, respectively. The transcriptional response data of MCF7 include 3,628 gene microarrays for 1,198 single-dose drugs, 96 multiple-dose drugs and 1, 390 drug-dose pairs. The transcriptional response data of PC3 have 2,017 gene microarrays for 1,150 single-dose drugs, 31 multiple-dose drugs and 1,215 drug-dose pairs. The transcriptional response data of HL60 comprise 1,406 gene microarrays for 1,061 single-dose drugs, 17 multiple-dose drugs, and 1,099 drug-dose pairs.

FIG. 6A, FIG. 6B, FIG. 6C, FIG. 6D and FIG. 6E illustrate the strategies used in the off-target repositioning method, CSB-BFRM, and the Quick Guide provides an overview of the key definitions and modeling components. For example, FIG. 6A shows the advantage of combining CSB and BFRM (Bild et al., 2006; Chang et al., 2009; Andrechek et al., 2008; Andrechek et al., 2009) to reposition drugs that cater not only the treatment response but also the expanded cancer signaling mechanisms, making it feasible for off-target repositioning for cancers. In FIG. 6B, the input to CSB-BFRM is a treatment-response matrix X (n×m) whose m columns correspond to the treated drugs and n rows correspond to the coding genes for the identified CSB proteins for the cancer type of interest. The statistical factor analysis, BFRM, decomposes the treatment-response matrix X into another two matrices, weight matrix A (n×k) and effect matrix Λ(k×m). A weight matrix, A (n×k), is a sparse matrix (most of elements are zero, as indicated by white color) whose columns define k signatures and their non-zero elements indicate which proteins are included in the signatures. BFRM imposes a sparse prior on the association of the genes to the signatures. Another matrix, P=(ρ₁,ρ₂, . . . , ρₖ), contains the (sparse) probabilities that each gene is associated with each factor. The cutoff for each element, P_{i j}, of P matrix was chosen as the mean of all the non-zero values in the P matrix. If P_{i j} is higher than the cutoff, the corresponding value, A_{i j}, of weight matrix A will be kept, and else, A_{i j} is set as zero. An effect matrix, Λ(k×m), demonstrates the effects of the m drugs imposed on the k signatures. BFRM model applies hierarchical priors for values of the non-zero elements in A and gets posterior via Markov Chain Monte Carlo (MCMC). MCMC analysis for the posterior simulation is implemented in a Gibbs sampling manner. The BFRM model is implemented by a software package, BFRM 2.0 (Bild et al., 2006; Chang et al., 2009). The number of signatures, k, was determined by an evolution algorithm in the BFRM 2.0 software.

In FIG. 6C, the repositioning profile definition takes advantages of the identification of the targetable signatures. If a drug's target information is available, the targetable signatures are defined by the non-zero weights at the rows of the targets across signatures of A. The proteins in each targetable signature are determined by the non-zero elements in each corresponding column of A. For each targetable signature, the total of the non-zero weights is used to evaluate the response of the signature to the drug. In Λ, the score corresponding to the row of the signature and the column of the drug shows the effect of the drug on the signature. The OTE that the drug imposes on the signature was defined as a weighted score obtained by multiplying the response of the signature to the drug by the effect of the drug on the signature. The repositioning profile was used to illustrate the OTEs of the drug on all of the signatures, in which the OTEs for the targetable signatures were defined as the weighted scores while those for the un-targetable signatures were zero.

Sometimes, the target information of a drug may be unavailable. Thus, a randomized process was designed to find these targetable signatures. In this randomized process, a number of proteins randomly chosen from the CSB proteins were considered as candidates for drug-targets. The hypothesis was that these drugs generate off-target effects (OTEs) on the CSB proteins even if they did not target the CSB proteins directly. The number of proteins chosen is determined by a random numerical value drawn from a uniform distribution between 1 and μ, where μ is the mean value of the targets for the drugs whose targets are known. The randomized process is repeated 1,000 times for those drugs whose targets are unknown to reduce the computational bias in the identification of their candidate targets or off-targets. Still some drugs have known drug-targets that are not included in the CSB protein set. These targets are led to the CSB proteins, using the shortest-paths in the protein-protein interaction network. The CSB proteins identified are considered as the targets or off-targets of these drugs.

To rank the activities of drugs, a single numerical value was proposed, termed the repositioning score, for each drug. In this study, since a number of drugs are known to be FDA-approved or undergoing clinical trials for breast cancer, prostate cancer, and promyelocytic leukemia, a supervised regression model was used to define the repositioning score (FIG. 6D). For other cancer types, the FDA-approval and clinical trial information may be unavailable. To apply the CSB-BFRM method to these cancer types, the supervised method should be replaced by an unsupervised data mining method, for example, clustering. The SVR algorithm is implemented in R, using the package “e1071.” All of the parameters are used as default except that the parameter ‘c’ for cross validation is set to be 5. Cross validation was specified as 5-fold.

Results

CSBs Expand the Signaling Proteins to Cancer Proteins

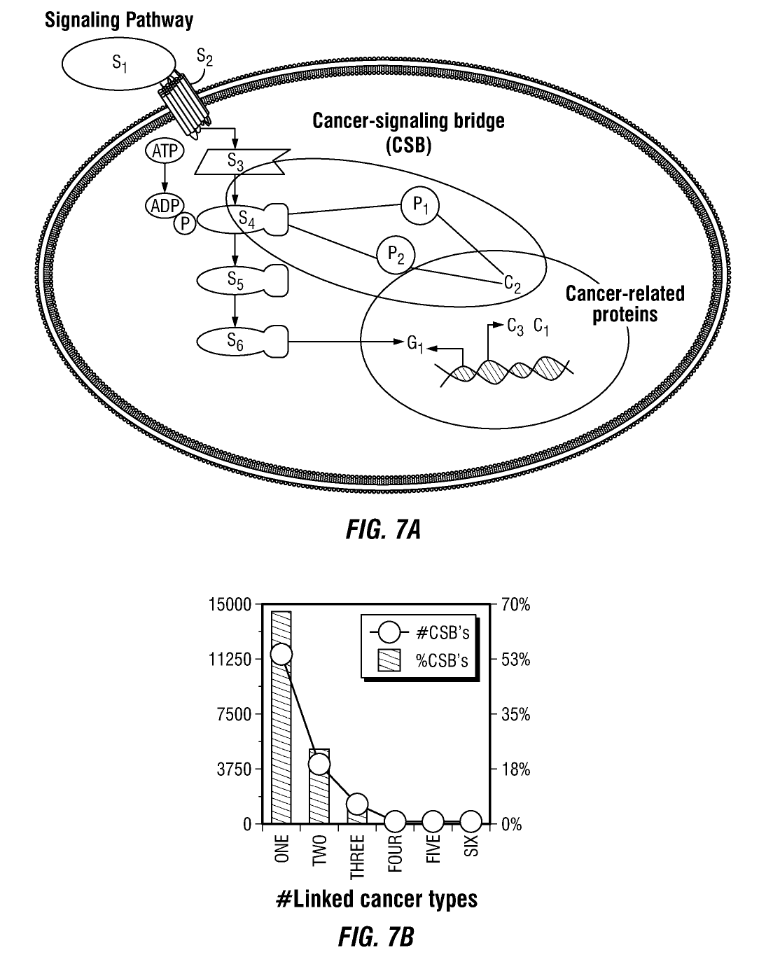

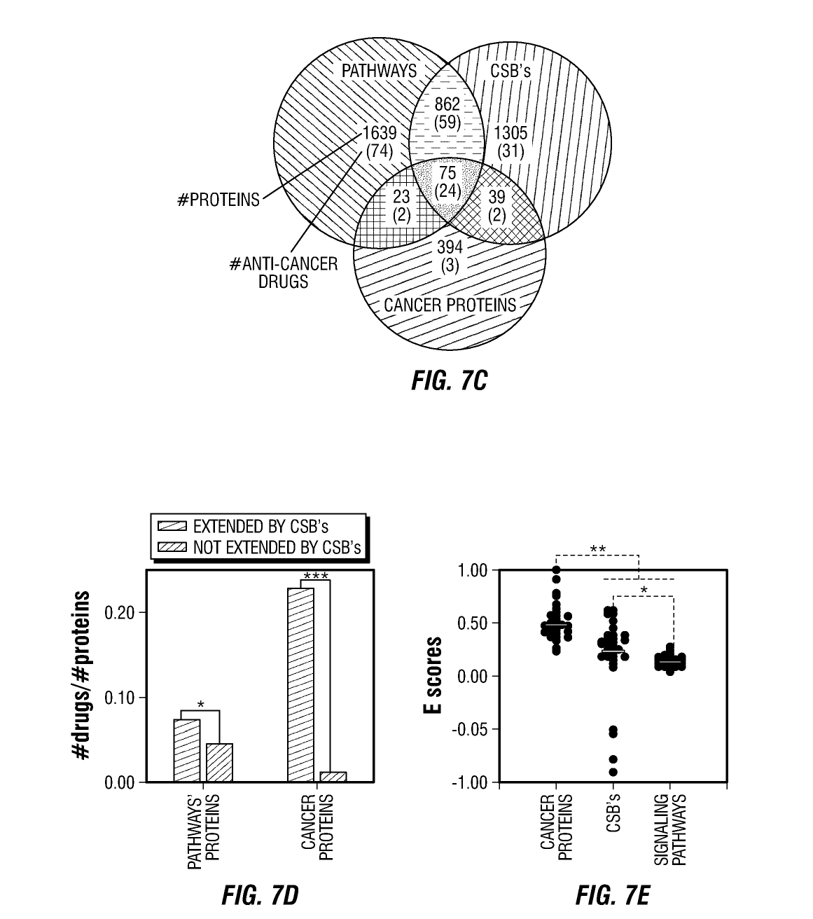

To investigate the off-target drug repositioning for cancers, new network elements, CSBs, were introduced that could be exploited to extend the known canonical signaling pathways (Schaefer, 2009; Izmailov et al., 2001) to the proteins whose coding genes have a close relationship with cancer genetic disorders (Hamosh et al., 2005; Yildirim et al., 2007), for short, cancer proteins (FIG. 7A). CSBs are the instances of network motifs (Milo et al., 2002; Jin et al., 2007; Shen-Orr et al., 2002), or building blocks, of the protein interaction networks (Kerrien et al., 2007; Xenarios et al., 2002; Chatr-aryamontri et al., 2007; Mewes et al., 2002; Breitkreutz et al., 2008) ENREF 21 ENREF 22 ENREF 23.

Besides being able to link many previously unrelated cancer proteins to a known signaling pathway of interest, CSBs have the following four characteristics that determine their important role in off-target drug repositioning: i) CSBs are significantly enriched in the connections between oncogenic signaling pathways and cancer proteins; ii) most CSBs, nearly 70%, are not shared by multiple types of cancers but are specific to one cancer type (FIG. 7B); iii) signaling proteins and cancer proteins linked by CSBs are significantly more likely to be targeted by known anti-cancer drugs (FIG. 7D); and iv) although most known anti-cancer drugs select the proteins in signaling pathways as their targets (FIG. 7C), they still generate relatively high effects, transmitted by CSBs, onto cancer proteins (FIG. 7E).

Application Of CSB-BFRM to Cancer Transcriptional Response Data

The off-target repositioning method, CSB-BFRM described herein was applied to three cancer transcriptional response datasets of MCF7 breast cancer cell line, PC3 prostate cancer cell line, and HL60 promyelocytic leukemia cell line. The inputs and outputs of CSB-BFRM were defined and the ability of CSB-BFRM to predict the activities of FDA-approved drugs and clinical trial drugs for breast cancer, prostate cancer, and promyelocytic leukemia was examined. The identified off-targets and OTEs were then used to explain the potential mechanisms of action of the repositioned drugs.

Performance of Repositioning Prediction

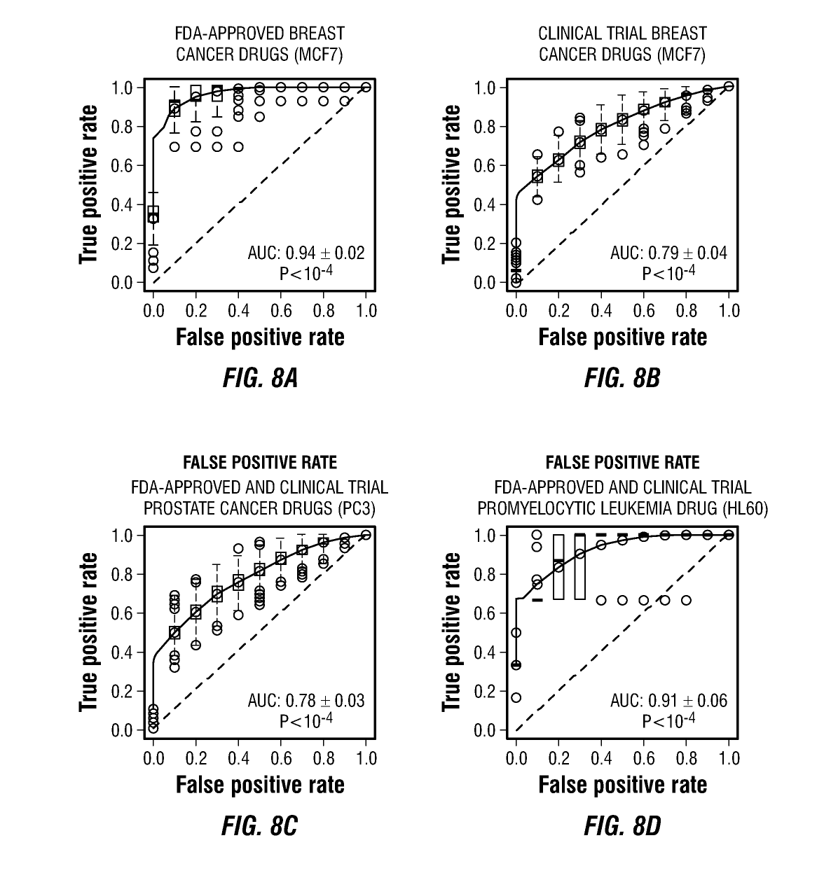

To evaluate the performance of CSB-BFRM in prediction of drug activities based on the identified repositioning profiles of drugs, the Receiver Operating Characteristic (ROC) method was employed. The area under the ROC Curve (AUC) illustrates how useful the repositioning profiles are for prediction of the known data of FDA-approval and clinical trials information. In FIG. 8A and FIG. 8B, it was shown that the ROC curves for the predictions on the activities of FDA-approved and clinical trial breast cancer drugs. The AUCs for the ROCs in FIG. 8A were 0.94±0.02 (P<10⁻⁴, Fisher's exact two-tailed test) and those for the ROCs in FIG. 8B were 0.79±0.04 (P<10⁻⁴, Fisher's exact two-tailed test). Since the FDA-approval information for prostate cancer and promyelocytic leukemia is limited, it was merged with clinical trial information in order to do the repositioning predictions.

The performance of the prediction on the FDA-approved and clinical trial prostate cancer drugs is indicated by the ROC curve shown in FIG. 8C. The AUC of the ROC curve in FIG. 8C is 0.78±0.03 (P<10⁻⁴, Fisher's exact two-tailed test). The ROC curve for the prediction on the FDA-approved and clinical-trial promyelocytic leukemia drugs is shown in FIG. 8D and its AUC was 0.91±0.06 (P<10⁻⁴, Fisher's exact two-tailed test). These results indicated that the activities of the FDA-approved and clinical trials drugs for breast cancer, prostate cancer, and promyelocytic leukemia were accurately predicted by the CSB-BFRM method described herein.

For repositioning on the MCF7 breast cancer cell line, the first 22 drugs with the highest repositioning scores are listed in Table 1. These first 22 drugs predict all 14 FDA-approved drugs (with drug dosages) from the 1,390 drugs (P<10⁻¹⁰, Hypergeometric test). The relatively small numbers of drugs with highest repositioning scores predict the FDA-approved drugs and clinical trial drugs for prostate cancer and promyelocytic leukemia (PC3: P<10⁻⁴, Hypergeometric test; HL60: P<10⁻², Hypergeometric test).

OTEs and Off-Targets

Eight pairs of drug-doses were examined with relatively high repositioning scores repositioned for MCF7 breast cancer cell line. The eight drug-dose pairs are raloxifene at 0.1 μM and 7.8 μM, tamoxifen at 1 μM and 7 μM, paclitaxel at 4.6 μM and 1 μM, and fulvestrant at 1 μM and 0.01 μM. Off-targets were identified for each of the four repositioned drugs. To remove the redundant off-targets with relatively lower OTEs, the mean of the absolute values, |OTE|, was used of all off-targets as a threshold, δ. Those off-targets, whose OTEs were higher than the threshold δ or lower than −δ were chosen for the following analysis. Gene set enrichment analysis (GSEA) (Subramanian et al., 2005) was performed on the off-targets of each drug. The off-targets of each drug were significantly enriched in two important cellular functions, cell cycle (P<10⁻⁵, Hypergeometric test) and apoptosis of cells (P<10⁻²⁶, Hypergeometric test). The enrichment P-values for all of the eight drugs were also determined. Pathway analysis was performed on the identified off-targets, using IPA (Ingenuity Pathway Analysis) software. Subsequently, two important signaling pathways related to cell cycle and apoptosis were identified, namely, cell cycle G1/S checkpoint and p53 signaling pathways.

Mechanisms of Repositioned Drugs

The Rb-dependent repression of E2F-mediated transcription (Sherr et al., 2002) is the key to understanding the mechanisms of the eight repositioned drug-dose pairs. The signal cascade was given as the following:

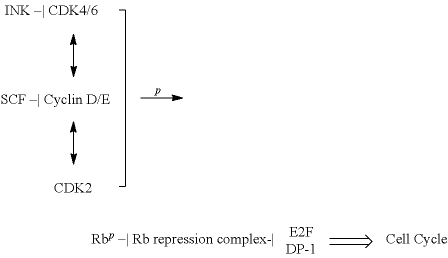

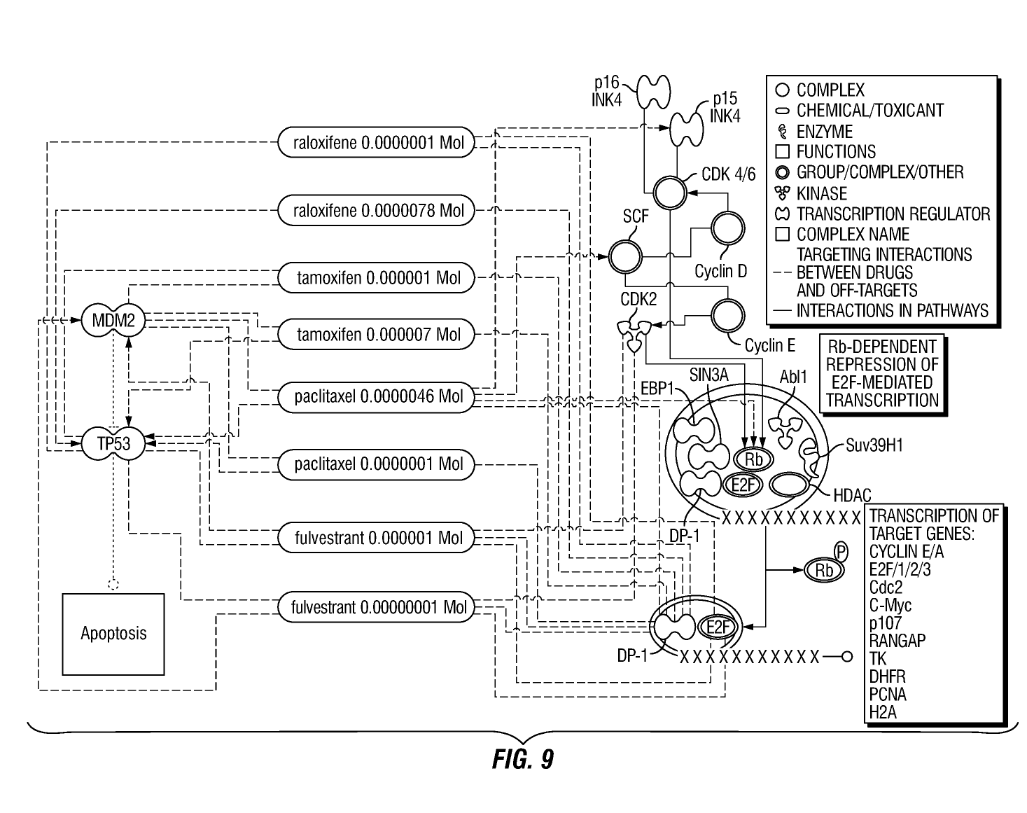

The OTEs and off-targets of the eight repositioning drug-dose pairs for this signaling cascade were determined. To better illustrate the drugs' effects on the signaling cascade, their OTEs and off-targets are also displayed in FIG. 9. All eight drug-dose pairs inhibit the core part of the signaling cascade, the heterodimer of E2F and DP-1. The inhibition of either E2F or DP-1 ensures that the gene expression is repressed even if Rb is phosphorylated. Still, some drug-dose pairs targeting on other parts of the signaling cascade enforce the transcriptional repression. Paclitaxel at 4.6 μM has a relatively high positive OTE (higher than 0) on the RBL1 protein (a member of the Rb protein family), which increases the expression of RBL1 protein and strengthens the recruitment of HDACs and other nuclear factors to repress gene expression. Paclitaxel at 4.6 μM also has positive OTEs on the INK4 (p15) and SCF proteins, which enhances the inhibition of CDK4/6 and Cyclin D/E as well as phosphorylation of Rb, so that the association of Rb family members with both HDACs and E2Fs are enhanced and gene expression is repressed. Fulvestrant, at 1 μM and 0.01 μM, had negative OTEs (i.e., <0) on the kinase CDK2, and decreased its expression, which in turn, reduced phosphorylated Rb and enhanced the Rb-dependent repression of E2F-mediated transcription. Thus, by various means, these drugs enforce the transcriptional repression of key cell cycle genes.

Consistency with Dose-Response Curves

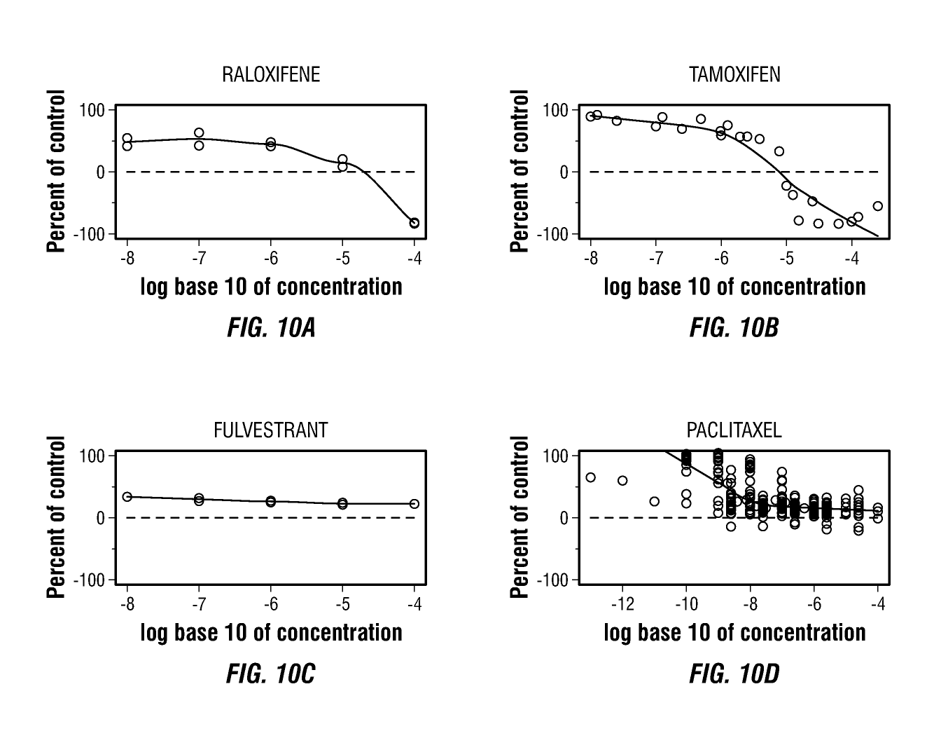

Dose-response data derived from the Developmental Therapeutics Program (DTP) of NCI/NIH (Doh et al., 2003) were used to validate the new method. Analysis of the dose-response curves for raloxifene, tamoxifene, paclitaxel, and fulvestrant (FIG. 10A, FIG. 10B, FIG. 10C, and FIG. 10D) showed that all four drugs with considered dosages (i.e., <10 μM) significantly inhibited cell growth. This result was consistent with the predicted OTEs enhancing the Rb-dependent repression of E2F-mediated transcription of the key genes for cell cycle progression.

The four repositioned drugs not only generated OTEs on the cell cycle G1/S checkpoint signaling pathway but also impose OTEs on the p53 signaling pathway (FIG. 9). The OTEs on the p53 signaling pathway were helpful in understanding why raloxifene, tamoxifen, and paclitaxel induce apoptosis at higher dosages, yet fulvestrant does not induce any cell death on MCF7 (FIG. 10A, FIG. 10B, FIG. 10C, and FIG. 10D). Comparing the OTEs of raloxifene at lower and higher dosages, these two OTEs are opposite to each other. At the lower dosage, the negative OTE decreases TP53 and blocks apoptosis while at the higher dosage it increases apoptotic cell death. This was also seen with tamoxifen. On the other hand, paclitaxel was predicted to increase the expression of TP53, and also induce apoptosis of cells at both lower and higher dosages. Several studies involving paclitaxel with dosages between 10⁻⁷M and 10⁻⁵M also induced cell death. In contrast, fulvestrant decreased the expression of TP53 at both lower and higher dosages, and could not induce apoptosis of cells at any of the considered dosages.

### Discussion

In summary, the present example provides a new computational method for off-target drug repositioning using cancer transcriptional response data before and after treatment. Facilitated by the new network elements, CSBs, it was shown that the new method, CSB-BFRM, performed well in repositioning drugs for specific cancer types. In particular illustrative examples, the CSB-BFRM method described herein was shown to be highly effective in predicting the activities of both FDA-approved and clinical-trial drugs for breast cancer, prostate cancer, and promyelocytic leukemia, using the corresponding transcription response datasets. The predicted OTEs and off-targets also helped to better explain the mechanisms of action of the repositioned drugs.

The repositioning list for MCF7 breast cancer cell line included all of the FDA-approved breast cancer drugs targeting on ER, which appear in the 1,390 drug set. These drugs were raloxifene at 0.1 μM and 7.8 μM, tamoxifen at 1 μM and 7 μM, fulvestrant at 1 μM and 0.01 μM, and estradiol at 4.6 μM and 1 μM. The repositioning results were consistent with the fact that MCF7 was an ER+ breast cancer cell line. Raloxifene and tamoxifen are selective estrogen-receptor modulators (SERMs) (Riggs et al., 2003). These SERMs function as pure antagonists when acting through estrogen receptor β on genes containing estrogen response elements but can function as partial agonists when acting on them through estrogen receptor α. The repositioning results for raloxifene and tamoxifen were consistent with the “partial agonist” property of raloxifene and tamoxifen. These two drugs generated higher effects at lower dosages. Raloxifene at 0.1 μM had a higher repositioning rank than raloxifene at 7.8 μM, and tamoxifen at 1 μM had a higher repositioning rank than tamoxifen at 7 μM

The identified off-targets and OTEs display the complexity of drugs' activities. On one hand, some drugs at higher dosages have their own specific off-targets or OTEs. In the repositioning for the MCF7 breast cancer cell line, paclitaxel at 4.6 μM has extra positive OTEs on the RBL1 protein, Rb's kinase (CDK2), the cyclin proteins' inhibitor (SCF), and the inhibitor of kinase CDK4/6, INK4 (p15), which are absent at the lower dosage. These OTEs ensure that paclitaxel can strengthen the transcription repression on the key genes regulating the cell cycle. On the other hand, at different dosages, the same drug would generate different effects on its specific off-targets and signaling pathways. For example, at the higher dosages, raloxifene and tamoxifen have positive OTEs on p53 protein while exhibiting negative OTEs on p53 protein at the lower dosages. Since the complexity of drugs' activities is not easily explained by “on-target” studies, the OTEs on the downstream signaling proteins have to be identified and linked to transcription, rather than simple analysis of the effects on known drug-targets.

BFRM played a significant role in recognizing the OTEs of the repositioned drugs in the present method. It factorizes the response (fold-change of expression) of a molecule into different component values according to the latent factors (signatures). The CSB-BFRM recognizes the essential latent factors (targetable signatures) and factorized component values (OTEs) for these signatures. For the repositioning on the MCF7 breast cancer cell line, the original response (fold-change) was compared on off-targets in cell cycle G1/S checkpoint and p53 signaling pathways with the recognized OTEs on these targets. The data scale was changed; fold-changes of the molecules were between 0.4 and 1.6 while OTEs are between 0.10 and 0.10. The factorized OTEs permitted easy recognition of positive and negative effects. For instance, all of the original fold-changes of tamoxifen at 1 μM are higher than 1, while the OTEs are between 0.05 and 0.05. If the original fold-changes were used, the difference between OTEs on the heterodimer of E2F and DP-1 (negative) and those for p53 (positive) could not be discriminated. The recognized OTEs were better in reflecting the mechanism of action of repositioned drugs.

The proposed off-target drug repositioning method, CSB-BFRM, takes advantage of the availability of disease-specific prior knowledge. For example, the definition of CSBs as shown in FIG. 6A and FIG. 6B needs the prior knowledge of the cancer genes that have genetic disorders associated with the cancer type of interest. Although CSB-BFRM analysis may be less effective in repositioning drugs for rare cancer types (since as prior knowledge is often unavailable), the method will likely become more robust in such analyses given the rapid development of next generation sequencing, and an increase in genetic mutation data.

### REFERENCES

The following references, to the extent that they provide exemplary procedural or other details supplementary to those set forth herein, are specifically incorporated herein in their entirety by express reference thereto:
Ande S R, Chen J, Maddika S “The ubiquitin pathway: an emerging drug target in cancer therapy,” Eur. J. Pharmacol., 625:199-205 (2009).
Andersson M K and Aman P “Proliferation of Ewing sarcoma cell lines is suppressed by the receptor tyrosine kinase inhibitors gefitinib and vandetanib,” Cancer Cell Int., 8:1 (2008).
Andrechek E R, Cardiff R D, Chang J T, Gatza M L, Acharya C R, Potti A, et al., “Genetic heterogeneity of Myc-induced mammary tumors reflecting diverse phenotypes including metastatic potential,” Proc. Natl. Acad. Sci. USA, 106:16387-92 (2009).
Andrechek E R, Mori S, Rempel R E, Chang J T, Nevins J R., “Patterns of cell signaling pathway activation that characterize mammary development,” Development, 135:2403-13 (2008).
Ashburn T T and Thor K B, “Drug repositioning: identifying and developing new uses for existing drugs,” Nat. Rev. Drug Discov., 3:673-83 (2004).
Bentires-Alj M, Kontaridis M I, Neel B G “Stops along the RAS pathway in human genetic disease,” Nat. Med., 12:283-285 (2006).
Biard D S, Miccoli L, Despras E, Frobert Y, Creminon C, et al., “Ionizing radiation triggers chromatin-bound kin17 complex formation in human cells,” J. Biol. Chem., 277:19156-19165 (2002).
Bild A H, Yao G, Chang J T, Wang Q, Potti A, Chasse D, et al., “Oncogenic pathway signatures in human cancers as a guide to targeted therapies,” Nature, 439:353-7 (2006).
Bolstad B M, Irizarry R A, Astrand M, Speed T P, “A comparison of normalization methods for high density oligonucleotide array data based on variance and bias,” Bioinformatics, 19:185-193 (2003).
Bos P D, Zhang X I I, Nadal C, Shu W, Gomis R R, et al., “Genes that mediate breast cancer metastasis to the brain,” Nature, 459:1005-1009 (2009).
Breitkreutz B J, Stark C, Reguly T, Boucher L, Breitkreutz A, Livstone M, et al., “The BioGRID Interaction Database: 2008 update,” Nucl. Acids Res., 36:D637-40 (2008).
Campillos M, Kuhn M, Gavin A C, Jensen L J, Bork P., “Drug target identification using side-effect similarity,” Science, 321:263-6 (2008).
Chang J T, Carvalho C, Mori S, Bild A H, Gatza M L, Wang Q, et al., “A genomic strategy to elucidate modules of oncogenic pathway signaling networks,” Mol. Cell., 34:104-14 (2009).
Chang M, Lee C H, Luo D D, Krupa A, Fraser D, et al., “Polarity of response to transforming growth factor-beta in proximal tubular epithelial cells is regulated by β-catenin,” J. Biol. Chem., 282:28639-28647 (2007).
Chatr-aryamontri A, Ceol A, Palazzi L M, Nardelli G, Schneider M V, Castagnoli L, et al., “MINT: the Molecular INTeraction database,” Nucl. Acids Res., 35:D572-4 (2007).
Creighton C J, Li X, Landis M, et al., “Residual breast cancers after conventional therapy display mesenchymal as well as tumor-initiating features,” Proc. Natl. Acad. Sci. USA, 106:13820-5, 2009.
Creighton C J, Massarweh S, Huang S, Tsimelzon A, Hilsenbeck S G, Osborne C K, et al. “Development of resistance to targeted therapies transforms the clinically associated molecular profile subtype of breast tumor xenografts,” Cancer Res., 68:7493-501 (2008).
Doh H, Roh S, Lee K W, Kim K. “Response of primed human PBMC to synthetic peptides derived from hepatitis B virus envelope proteins: a search for promiscuous epitopes,” FEMS Immunol. Med. Microbiol., 35:77-85 (2003).
Downey T, “Analysis of a multifactor microarray study using Partek genomics solution,” Methods Enzymol., 411:256-270 (2006).
Efroni S, Schaefer C F, Buetow K H, “Identification of key processes underlying cancer phenotypes using biologic pathway analysis,” PLoS One, 2(5):e425 (2007).
Feyen O, Lueking A, Kowald A, Stephan C, Meyer H E, Gobel U, et al., “Off-target activity of TNF-alpha inhibitors characterized by protein biochips,” Anal. Bioanal. Chem., 391:1713-20 (2008).
Gautschi O, Tschopp S, Olie R A, Leech S H, Simoes-Wust A P, et al., “Activity of a novel bcl-2/bcl-xL-bispecific antisense oligonucleotide against tumors of diverse histologic origins,” J. Natl. Cancer Inst., 93(6):463-471 (2001).
Hamosh A, Scott A F, Amberger J S, Bocchini C A, McKusick V A, “Online Mendelian Inheritance in Man (OMIM), a knowledgebase of human genes and genetic disorders,” Nucl. Acids Res., 33:D514-517 (2005).
Iorio F, Bosotti R, Scacheri E, Belcastro V, Mithbaokar P, Ferriero R, et al. “Discovery of drug mode of action and drug repositioning from transcriptional responses,” Proc. Natl. Acad. Sci. USA, 107:14621-6 (2010).
Izmailov A, Yager T D, Zaleski H, Darash S. “Improvement of base-calling in multilane automated DNA sequencing by use of electrophoretic calibration standards, data linearization, and trace alignment,” Electrophoresis, 22:1906-14 (2001).
Jin G, Zhang S, Zhang X S, Chen L. “Hubs with network motifs organize modularity dynamically in the protein-protein interaction network of yeast,” PLoS One, 2:e1207 (2007).
Keiser M J, Hert J. “Off-target networks derived from ligand set similarity,” Methods Mol. Biol., 575:195-205 (2009).
Keiser M J, Setola V, Irwin J J, Laggner C, Abbas A I, Hufeisen S J, et al. “Predicting new molecular targets for known drugs,” Nature, 462:175-81 (2009).
Kerrien S, Alam-Faruque Y, Aranda B, Bancarz I, Bridge A, Derow C, et al. “IntAct—open source resource for molecular interaction data,” Nucl. Acids Res., 35:D561-5 (2007).
Knauf J A and Fagin J A, “Role of MAPK pathway oncoproteins in thyroid cancer pathogenesis and as drug targets,” Curr Opin Cell Biol., 21:296-303 (2009).
Konstantiniclou G, Bey E A, Rabellino A, Schuster K, Mara M S, et al., “Dual phosphoinositide 3-kinase/mammalian target of rapamycin blockade is an effective radiosensitizing strategy for the treatment of non-small cell lung cancer harboring K-RAS mutations,” Cancer Res., 69:7644-7652 (2009).
Lamb J, Crawford E D, Peck D, Modell J W, Blat I C, Wrobel M J, et al., “The Connectivity Map: using gene-expression signatures to connect small molecules, genes, and disease,” Science, 313:1929-35 (2006).
Lamb J, Ramaswamy S, Ford H L, Contreras B, Martinez R V, et al., “A mechanism of cyclin 131 action encoded in the patterns of gene expression in human cancer,” Cell, 114:323-334 (2003).
Lamb J. “The Connectivity Map: a new tool for biomedical research,” Nat. Rev. Cancer, 7:54-60 (2007).
Li X, Lewis M T, Huang J, et al., “Intrinsic resistance of tumorigenic breast cancer cells to chemotherapy,” J. Natl. Cancer Inst., 100:672-9 (2008).
Lu Q, Longo F M, Zhou H, Massa S M, Chen Y H, “Signaling through Rho GTPase pathway as viable drug target,” Curr Med. Chem., 16:1355-1365 (2009).
Marks F, Klingmuller U, Muller-Decker K, Cellular Signal Processing: An Introduction to the Molecular Mechanisms of Signal Transduction, Garland Science, New York (2009).
Mewes H W, Frishman D, Guldener U, Mannhaupt G, Mayer K, Mokrejs M, et al., “MIPS: a database for genomes and protein sequences,” Nucl. Acids Res., 30:31-4 (2002).
Mid A H, Yao G, Chang J T, Wang Q, Potti A, et al., “Oncogenic pathway signatures in human cancers as a guide to targeted therapies,” Nature, 439:353-357 (2006). Miller M A., “Chemical database techniques in drug discovery,” Nat. Rev. Drug Discov., 1:220-7 (2002).
Milo R, Shen-Orr S, Itzkovitz S, Kashtan N, Chklovskii D, Alon U. “Network motifs: simple building blocks of complex networks,” Science, 298:824-7 (2002).
Riggs B L, Hartmann LC. “Selective estrogen-receptor modulators—mechanisms of action and application to clinical practice,” N Engl. J. Med., 348:618-29 (2003).
Sardana D, Zhu C, Zhang M, Gudivada R C, Yang L, Jegga A G. “Drug repositioning for orphan diseases,” Brief Bioinform., 12:346-56 (2011).
Schaefer C F, Anthony K, Krupa S, Buchoff J, Day M, Hannay T, et al., “PID: the Pathway Interaction Database,” Nucl. Acids Res., 37:D674-9 (2009).
Shen-Orr S S, Milo R, Mangan S, Alon U. “Network motifs in the transcriptional regulation network of Escherichia coli,” Nat. Genet., 31:64-8 (2002).
Sherr C J, McCormick F., “The RB and p53 pathways in cancer,” Cancer Cell, 2:103-12 (2002).
Sirota M, Dudley J T, Kim J, Chiang A P, Morgan A A, Sweet-Cordero A, et al., “Discovery and preclinical validation of drug indications using compendia of public gene expression data,” Sci. Transl. Med., 3:96ra77 (2011).
Subramanian A, Tamayo P, Mootha V K, Mukherjee S, Ebert B L, Gillette M A, et al., “Gene set enrichment analysis: a knowledge-based approach for interpreting genome-wide expression profiles,” Proc. Natl. Acad. Sci. USA, 102:15545-50 (2005).
Tartaglia M, Gelb B D, “Noonan syndrome and related disorders: genetics and pathogenesis,” Annu. Rev. Genomics Hum. Genet., 6:45-68 (2005).
Terzis A J, Thorsen F, Heese O, Visted T, Bjerkvig R, et al., “Proliferation, migration and invasion of human glioma cells exposed to paclitaxel (Taxol) in vitro,” Br. J. Cancer, 75:1744-1752 (1997).
Wee S. Jagani Z, Xiang K X, Loo A, Dorsch M, et al., “P13K pathway activation mediates resistance to MEK inhibitors in KRAS mutant cancers,” Cancer Res., 69:4286-4293 (2009).
Wishart D S, Knox C, Guo A C, Cheng D, Shrivastava S. et al., “DrugBank: a knowledgebase for drugs, drug actions and drug targets,” Nucl. Acids Res., 36:D901-D906 (2008).
Xenarios I, Salwinski L, Duan X J, Higney P, Kim S M, Eisenberg D. “DIP, the Database of Interacting Proteins: a research tool for studying cellular networks of protein interactions,” Nucl. Acids Res., 30:303-5 (2002).
Yan W, Liu G, Scoumanne A, Chen X “Suppression of inhibitor of differentiation 2, a target of mutant p53, is required for gain-of-function mutations,” Cancer Res., 68:6789-6796 (2008).
Yarde D N, Oliveira V, Mathews L, Wang X, Villagra A, et al., “Targeting the Fanconi anemialBRCA pathway circumvents drug resistance in multiple myeloma,” Cancer Res., 69:9367-9375 (2009).
Yildirim M A, Goh K I, Cusick M E, Barabasi A L, Vidal M. “Drug-target network,” Nat. Biotechnol., 25:1119-26 (2007).
Zhigang Z and Wenlu S “Flutamide reduced prostate cancer development and prostate stem cell antigen mRNA expression in high grade prostatic intraepithelial neoplasia,” Int. J Cancer, 122:864-870 (2008).

All of the compositions and methods disclosed and claimed herein can be made and executed without undue experimentation in light of the present disclosure. While the compositions and methods of this invention have been described in terms of exemplary embodiments, it will be apparent to those of ordinary skill in the art that variations may be applied to the composition, methods and in the steps or in the sequence of steps of the method described herein without departing from the concept, spirit and scope of the invention. More specifically, it will be apparent that certain agents that are both chemically- and physiologically related may be substituted for the agents described herein while the same or similar results would be achieved. All such similar substitutes and modifications apparent to those of ordinary skill in the art are deemed to be within the spirit, scope and concept of the invention as defined herein.

What is claimed is:

1. A method for repurposing a known pharmaceutical compound to a new treatment protocol, the method comprising: a) identifying a pharmaceutical compound, wherein the pharmaceutical compound is an untested compound, a compound that has failed in clinical development, or a drug that has been approved for use in another distinct treatment protocol; b) creating a computer model for one or more cancer signaling bridges; c) performing computer simulations using the computer model with data obtained from one or more in vitro or in vivo assays, one or more animal models of disease, or one or more pre-clinical or clinical trials of the pharmaceutical compound in at least a first human subject; and d) identifying a new treatment protocol for the known pharmaceutical compound based upon the results obtained from c).

2. The method of claim 1, wherein the new treatment protocol is based on at least one component selected from the group consisting of a new indication, a new drug, a new dosing regimen, a new drug combination, a new patient population, and the use of one or more biomarkers represented in the computer model to differentiate between responders and non-responders in the patient population.

3. The method of claim 2, wherein the new indication is the treatment of cancer or a hematological disorder.

4. The method of claim 3, wherein the new indication is the treatment of triple negative breast cancer.

5. The method of claim 3, wherein the known pharmaceutical compound exhibits anti-breast tumor initiating cell activity, and the new indication is breast cancer.

6. The method of claim 3, wherein treatment of the new indication with the repurposed drug reduces drug toxicity, increases drug efficacy, or both, in a recipient patient as compared to an existing drug already approved for treatment of said indication.

7. The method of claim 1, wherein the known pharmaceutical compound is a drug approved by the FDA for administration to a human.

8. The method of claim 7, wherein the drug is approved for at least one anti-cancer indication other than breast cancer.

9. The method of claim 8, wherein the drug is selected from the group consisting of L-glutamic acid, L-proline, sorafenib, rifabutin, adenosine, simvastatin, naltrexone, clodronate, auranofin, thalidomide, atorvastatin, arsenic trioxide, saquinavir, glucosamine, procaterol, clenbuterol, pranlukast, aminone, sunitinib, dasatinib, and chloroquine.

10. The method of claim 1, wherein the repurposing involves “on-target” repositioning of the known pharmaceutical compound.

11. The method of claim 1, wherein the repurposing involves “off-target” repositioning of the known pharmaceutical compound.

12. The method of claim 1, wherein the data obtained from one or more in vitro or in vivo assays includes data obtained from one or more gene expression profiles, one or more protein expression profiles, one or more protein:protein interaction networks, one or more signaling pathways, one or more cancer signaling bridges, or any combination thereof.

13. The method of claim 1, wherein the data obtained from one or more in vitro or in vivo assays is indicative of drug targeting to one or more of proteins selected from the group consisting of TNF, KDR, IKBKB, Notch, hedgehog, wnt, P13K, FYN, SLC25A6, HSP90AA1, and EPRS, or to one or more genes that encode the one or more proteins, or any combination thereof.

14. The method of claim 1, wherein the known pharmaceutical compound has a low or insignificant off target effects on one or more of the gene(s) or protein(s) downstream in at least one of the metabolic or signaling pathway(s) for the new indication.

15. The method of claim 1, wherein the computer model uses a bioinformatic systems model-based approach that exploits one or more of the following: a) known protein-protein interactions, b) one or more steps in a signaling pathway that controls one or more steps in the metabolic pathway giving rise to the new indication being treated, and c) extant pharmacological information to increase the likelihood of identified a compound that is effective in treating the new indication.

16. The method of claim 1, wherein the computer model of the new indication is used to design a pre-clinical trial or clinical trial for testing the effectiveness of the repurposed pharmaceutical compound in vivo.

17. The method of claim 1, wherein the data is contained within a centralized computer database that is accessible by the computer model.

18. A method for identifying a candidate drug for the treatment of a selected disease or condition, the method comprising:
1) selecting a disease or condition to be treated;
2) creating a dynamic model of the disease;
3) performing in silico screening of a population of drugs, one or more of which is approved for other diseases or conditions that target one or more biological pathways implicated in the model to identify one or more candidate drugs;
4) testing the one or more candidate drugs selected in 3), either alone or in one or more combination(s) of drugs, in a biological model of the disease; and
5) identifying from within the candidate drugs tested in 4), one or more bioeffective drugs, or combinations thereof, suitable for the treatment of the selected disease or condition.

19. The method of claim 18, wherein the disease or condition is a neurologic disease or disorder; a psychiatric disease or disorder; an autoimmune disease or disorder; a cardiovascular disease or disorder; a metabolic condition, dysfunction, or disease; a neuropathic dysfunction, disorder, or disease; a hematological disease or disorder; a neuromuscular disease or dysfunction; or one or more hyperproliferative disorders such as cancer.

20. The method of claim 19, wherein the disease is breast cancer.

21. The method of claim 19, wherein the disease is caused by breast tumor initiating cells.

22. The method of claim 18, wherein the dynamic model of the disease or condition is created by compiling experimental data that describes or characterizes the disease or condition phenotype at a genomic, a biochemical, a cellular or an organismal level, within the body of an animal susceptible to the disease or condition.

23. The method of claim 18, wherein the model is obtained by combining the available information into a logically supported pathway involved in disease genesis and progression.

24. The method of claim 18, further comprising an additional step of identifying one or more pathways of the model of the disease or condition that could be modulated or blocked by the action of one or more identified candidate drugs.

25. The method of claim 18, wherein step 3) includes the identification or selection of one or more approved drugs that are known to interact with at least one target or with at least one step in a metabolic or signaling pathway that is included in, or that defines a key step in, the dynamic model of the disease or condition.

26. A method of producing a pharmaceutical compound, or a combination of two or more pharmaceutical compounds, for treating a selected disease, dysfunction, or condition in an animal, the method comprising:
a) identifying a candidate drug or a combination of candidate drugs effective for treating the selected disease, dysfunction, or condition using the method of claim 18; and
b) producing the candidate drug or the combination of candidate drugs in a pharmaceutically acceptable form.

27. A computer program product comprising a computer readable media having instructions that allows a computer to implement a process comprising: a) identifying a pharmaceutical compound, the pharmaceutical compound corresponding to a drug that has failed in clinical development or an approved drug; b) creating a computer model for pharmacokinetics and pharmacodynamics of the drug is created based on data of effects of the drug administered in vitro or in vivo to determine the physiological effect of the drug on a disease; c) adjusting the computer model based upon results of computer simulations from at least one pre-clinical or at least one clinical trial; d) determining a new treatment protocol to salvage the failed drug or the approved drug based on the results of computer simulation results; and e) displaying the new treatment protocol in an output window.

28. A system for off-target repositioning of a pharmaceutical compound, the system comprising: identifying a pharmaceutical compound, wherein the pharmaceutical compound is a drug that has failed in clinical development or an approved drug; a computer model for pharmacokinetics and pharmacodynamics of the drug is created based on data of effects of the drug administered in vitro or in vivo to determine the physiological effect of the drug on a disease; a model adjustor that adjusts the computer model based upon results of computer simulations from at least one pre-clinical or at least one clinical trial; a treatment protocol generator that results in the off-target repositioning of the pharmaceutical to a new indication or the identification of a new treatment protocol for the repositioned compound.

29. The system of claim 28, wherein the new treatment protocol is based on at least one component selected from a new regimen, a new drug combination, a new disease or condition, a new patient population, or a use one or more biomarkers represented in the computer model to differentiate between responders and non-responders in the patient population.

30. The system of claim 28, wherein the computer model involves analysis of one or more cancer signaling bridges.

31. The system of claim 30, wherein the analysis of one or more cancer signaling bridges is useful in identifying candidate drugs for the treatment of cancer.

32. The system of claim 31, wherein identified candidate drugs are effective against breast cancer tumor initiating cells.
